(19) United States
(12) Patent Application Publication
(10) Pub. No.: US 2015/0342857 A1
(43) Pub. Date: Dec. 3, 2015

(54) COMPOSITIONS FOR STRAIGHTENING OR RELAXING HAIR

(71) Applicant: L'OREAL, Paris (FR)

(72) Inventors: Michael DEGEORGE, OLD BRIDGE, NJ (US); Mark BENN, Union, NJ (US)

(73) Assignee: L'OREAL, Paris (FR)

(21) Appl. No.: 14/290,423

(22) Filed: May 29, 2014

(51) Int. Cl. A61K 8/81; A61Q 5/06; A61K 8/49; A61K 8/60; A61K 8/19; A61K 8/41

(52) U.S. Cl. CPC A61K 8/8135; A61K 8/19; A61K 8/41; A61K 8/4973; A61K 8/602; A61Q 5/06; A61K 2800/20

(57) ABSTRACT

Disclosed herein is a composition for relaxing or straightening hair containing an alkaline material comprising hydroxide-containing compounds, a crosslinked copolymer of (meth)acrylic acid and/or (C1-C6)alkyl esters selected from a crosslinked (meth)acrylic acid/ethyl acrylate copolymer and a cross-linked anionic acrylate polymer, a cosmetically acceptable solvent, and optionally, thickening agents and surfactants. The compositions of the invention may be in the form of a gel that is clear in appearance or may be in the form of an emulsion when the compositions additionally contain a liquid fatty substance.

### FIELD OF THE INVENTION

The present application relates to compositions for use on keratinous substrates. In particular, it relates to compositions and methods for relaxing or straightening hair.

### BACKGROUND OF THE INVENTION

Cosmetic and personal care products for use on keratinous substrates such as skin and hair are available commercially in various forms, for example, as creams, lotions, gels, pastes, and powders. Regardless of the form, these products have to achieve and provide certain benefits and attributes such as efficaciousness, cosmeticity, desirable texture, stable formulations, and ease and convenience of use and application. Thus, in order to meet changing consumer needs and preferences, manufacturers of such products continuously seek to re-formulate and create new products with enhanced efficacy, while still remaining stable, safe to use, as well as improving the manufacture, transport, and storage of these products. In addition, manufacturers continue to test the use of new raw materials and ingredients or new product forms that would help deliver the desired attributes and properties with respect to viscosity, texture, stability and efficacy.

One area where manufacturers are always seeking to provide improved consumer and cosmetic products is in the area of hair styling. There are many techniques and compositions for styling or altering the appearance and shape of hair. In today's market, there is an increasing demand for the hair care products referred to as “hair relaxers” or “hair straighteners” which can relax or straighten curly or kinky hair, including wavy hair. Straightening or relaxing the curls of very curly hair may increase the manageability and ease of styling of such hair. Hair relaxers may either be applied in a hair salon by a professional or in the home by the individual consumer.

One type of composition that can be applied onto hair in order to change its shape and make it more manageable is an alkaline composition. Alkaline hair relaxing/straightening consists of hydrolysis of the keratin of the hair with various alkaline agents, such as inorganic hydroxides, for instance sodium hydroxide, or organic hydroxides, such as guanidine hydroxide, or organic amines. Hair relaxing/straightening products that employ sodium hydroxide or potassium hydroxide are also called lye-based products and products that use other alkaline agents such as lithium hydroxide, calcium hydroxide, organic hydroxides and other non-hydroxide compounds, for example, organic amines, generally fall under the category of no-lye products.

Attributes of hair relaxing/straightening products that can affect their performance and/or desirability to consumers include texture, form or appearance (liquid, cream, gel, lotion or powder), ease of application, viscosity or rheology, ease of use, storage, effect on the quality and feel of the hair, and safety. For example, hair relaxing and straightening products should have a viscosity such that they do not run or drip when applied onto the hair fibers so as to avoid contacting the skin with the product and to ensure that the alkaline or straightening agents in the product are deposited onto the hair fibers and remain on the fibers during the straightening/relaxation period.

While certain polymers can be added into cosmetic products in order to achieve a desired viscosity or rheology, the inclusion of polymers into high pH or highly alkaline systems such as hair relaxing/straightening products can pose formulation challenges because the polymers can become unstable in products in high pH compositions, leading to unstable formulas (e.g., phase separation) and/or formulas with undesirable viscosities, i.e, a thin consistency.

The present invention provides a composition having a pH of at least 9 and comprising an alkaline material comprising a hydroxide-containing compound, a crosslinked copolymer of (meth)acrylic acid and/or (C1-C6)alkyl esters, and a cosmetically acceptable solvent, wherein the crosslinked copolymer is preferably, fully or partially neutralized.

The present invention also provides a method for relaxing/straightening hair in an easy and efficacious manner, using the composition of the present invention.

It was surprisingly and unexpectedly discovered that the combination of the above-described ingredients result in a composition that is stable and has a viscosity that corresponds to a thick, non-drip, and homogeneous consistency that facilitates the ease of application of the composition onto hair fibers, thereby resulting in an effective means of relaxing or straightening hair.

### BRIEF SUMMARY OF THE INVENTION

The present invention relates to a composition containing,
(a) at least one alkaline material comprising at least one hydroxide-containing compound selected from alkali metal hydroxides, alkaline-earth metal hydroxides, transition metal hydroxides, and mixtures thereof;
(b) at least one crosslinked copolymer of (meth)acrylic acid and/or (C1-C6)alkyl esters selected from a crosslinked (meth)acrylic acid/ethyl acrylate copolymer, a cross-linked anionic acrylate polymer, and mixtures thereof; and
(c) a cosmetically acceptable solvent;
wherein the pH of the composition ranges from about 9 to about 14; and wherein the viscosity of the composition ranges from about 50 uD to about 90 uD.

For the above-described composition, the crosslinked copolymer (b) is preferably fully or partially neutralized by at least one neutralizing agent;

The invention also relates to a method of relaxing or straightening hair, the method comprising contacting hair with the above described composition; and 2) leaving the composition on the hair for a sufficient period of time to achieve a desired relaxation or straightening.

The composition of the present invention may additionally contain one of more of the following: at least one liquid fatty substance; a least one thickening agent; at least one surfactant chosen from anionic surfactants, nonionic surfactants, amphoteric surfactants, and mixtures thereof; and at least one auxiliary ingredient chosen from emulsifying agents, conditioning agents, moisturizing agents, shine agents, fillers, pigments, chelating agents, sequestering agents, fragrances, preservatives, stabilizers, and mixtures thereof.

In certain embodiments, after the composition of the present invention is applied onto hair, the composition is left to stand on the hair for a period of time ranging from about 5 to 60 minutes, and preferably, from 5 to 30 minutes, after which, the hair is rinsed, and optionally, dried.

In other embodiments, the composition is provided to the end user in a container such as a tube (e.g., a squeeze tube), a bottle, or a jar.

The compositions of the present invention are stable over time and they can be stored for several months without modification and with very little change or fluctuation in the viscosity and/or rheology and pH of the composition. When the composition of the present invention is in the form of an emulsion, the emulsion is stable and does not exhibit phase separation.

It is to be understood that both the foregoing general description and the following detailed description are exemplary and explanatory only, and are not restrictive of the invention.

### BRIEF DESCRIPTION OF THE DRAWING

FIG. 1 represents a photographic image of hair samples, showing the straightening/relaxing effects on hair imparted by the composition of the invention.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 5% to 10% of the indicated number.

“Keratinous substrates” as used herein, include, but are not limited to skin, lips, and keratin or keratinous fibers such as hair and eyelashes.

“Volatile”, as used herein, means having a flash point of less than about 100° C.

“Non-volatile”, as used herein, means having a flash point of greater than about 100° C.

“Active material” as used herein with respect to the percent amount of an ingredient or raw material, refers to 100% activity of the ingredient or raw material.

The term “clear” as used herein means transparent such that a person is able to see through the composition with their naked eye. The term “clear” as used herein also means that the composition does not exhibit phase separation. The term “clear” as used herein is not meant to encompass those compositions which a person cannot see through with their naked eye such as those which are pearlescent, frosted, hazy, opaque, or cloudy in appearance.

The expression “acrylic polymer” of “acrylic-containing polymer” is understood, for the purposes of the present invention, to mean a polymer that results from the polymerization of one or more monomers.

The compositions and methods of the present invention can comprise, consist of, or consist essentially of the essential elements and limitations of the invention described herein, as well as any additional or optional ingredients, components, or limitations described herein or otherwise useful.

In an embodiment, the present invention relates to a composition comprising:
(a) from about 1% to about 20% by weight of active material of at least one alkaline material comprising at least one hydroxide-containing compound selected from alkali metal hydroxides, alkaline-earth metal hydroxides, transition metal hydroxides, and mixtures thereof;
(b) from about 1% to about 5% by weight of active material of at least one crosslinked copolymer of (meth)acrylic acid and/or (C1-C6)alkyl esters selected from a crosslinked (meth)acrylic acid/ethyl acrylate copolymer, a cross-linked anionic acrylate polymer, and mixtures thereof; and
(c) a cosmetically acceptable solvent;
   all weights above being based on the total weight of the composition;
   wherein the crosslinked copolymer (b) is fully or partially neutralized by about 0.5% to about 3% by weight of at least one neutralizing agent;
   wherein the pH of the composition ranges from about 9 to about 14.

The viscosity of the above-described composition is preferably from about 50 uD to about 90 uD.

In another embodiment, the present invention relates to a composition comprising:
(a) from about 2% to about 6% by weight of active material of at least one alkaline material comprising at least one hydroxide-containing compound selected from sodium hydroxide, potassium hydroxide, lithium hydroxide, and mixtures thereof;
(b) from about 1% to about 5% by weight of active material of at least one crosslinked copolymer of (meth)acrylic acid and/or (C1-C6)alkyl esters selected from a crosslinked (meth)acrylic acid/ethyl acrylate copolymer, a cross-linked anionic acrylate polymer, and mixtures thereof; and
(c) a cosmetically acceptable solvent;
   all weights above being based on the total weight of the composition;
   wherein the crosslinked copolymer (b) is fully or partially neutralized by about 0.6% to about 1.2% by weight of at least one neutralizing agent;
   wherein the pH of the composition ranges from about 12.0 to about 13.6.

The viscosity of the above described composition is preferably, from about 50 uD to about 80 uD.

In an embodiment, the crosslinked copolymer of (meth)acrylic acid and/or (C1-C6)alkyl esters of the above-described compositions comprises a crosslinked (meth)acrylic acid/ethyl acrylate copolymer.

In another embodiment, the crosslinked copolymer of (meth)acrylic acid and/or (C1-C6)alkyl esters of the above-described compositions comprises a cross-linked anionic acrylate polymer.

In another embodiment, the cosmetically acceptable solvent of the above-described compositions comprises water or a water/organic solvent mixture.

In preferred embodiments, the above-described compositions of the present invention are in the form of an aqueous composition that is clear in appearance.

In another embodiment, the above-described compositions of the present invention may further comprise a liquid fatty substance selected from alkanes, such as C₆-C₁₆ alkanes, non-silicone oils of plant, mineral or synthetic origin, liquid fatty alcohols, liquid fatty acids and liquid esters of a fatty acid and/or of a fatty alcohol, or mixtures thereof. When the liquid fatty substance is present in an amount of at least about 10% by weight, preferably, from about 40% to about 60% by weight, based on the total weight of the composition, the composition of the present invention is in the form of an emulsion. A preferred liquid fatty substance of the present invention is mineral oil.

In an embodiment, the compositions of the present invention further comprise at least one thickening agent.

In another embodiment, the compositions of the present invention further comprise at least one surfactant.

In certain embodiments, the compositions of the present invention further comprise at least one auxiliary ingredient chosen from emulsifying agents, conditioning agents, moisturizing agents, shine agents, fillers, colorants, pigments, chelating compounds, sequestering agents, fragrances, preservatives, stabilizers, and mixtures thereof.

In another embodiment, the present invention relates to a method of relaxing or straightening hair, the method comprising the steps of contacting hair with any one of the above-described compositions; and leaving the composition on the hair for a sufficient period of time to achieve a desired relaxation or straightening.

It was surprisingly and unexpectedly discovered that the composition of the invention had a thick, smooth, non-drip, and homogenous texture/consistency and effectively relaxed or straightened hair.

It was also surprisingly and unexpectedly discovered that the composition of the invention was stable over time and retained the straightening or relaxing activity of the alkaline material such that when hair was contacted with the composition, the hair was effectively straightened or relaxed hair after the composition was allowed to stand on the hair for a period of time.

Furthermore, it was surprisingly and unexpectedly discovered that even when a liquid fatty substance, such as an oil, was present even at high levels in the composition of the present invention, the composition formed a stable and creamy and smooth emulsion that was easy to apply and spread on the hair, and did not easily drip or run off of the hair fibers.

The non-drip consistency of the compositions of the present invention is desirable because it helps prevent the compositions from coming in contact with and causing irritation on the skin or scalp.

The consistency/texture and non-drip attributes of the compositions can also be characterized in terms of their viscosities which were found to range from about 50 uD to about 90 uD.

### Alkaline Material

The present invention employs at least one alkaline material comprising at least one hydroxide-containing compound.

The at least one hydroxide-containing compound may be selected from alkali metal hydroxides, alkaline-earth metal hydroxides, transition metal hydroxides, and mixtures thereof.

In some embodiments, the at least one hydroxide-containing compound is selected from sodium hydroxide, potassium hydroxide, lithium hydroxide, calcium hydroxide, magnesium hydroxide, barium hydroxide, strontium hydroxide, manganese hydroxide, zinc hydroxide, and mixtures thereof.

In other embodiments, the at least one hydroxide-containing compound is selected from sodium hydroxide, potassium hydroxide, lithium hydroxide, calcium hydroxide, and mixtures thereof.

According to certain embodiments, the at least one hydroxide-containing compound is selected from sodium hydroxide, potassium hydroxide, and mixtures thereof.

According to other embodiments, the at least one hydroxide-containing compound is lithium hydroxide.

In yet other embodiments, the at least one hydroxide-containing compound is calcium hydroxide.

The amount of the at least one hydroxide-containing compound is preferably such that the composition has a pH of at least about 9, preferably ranging from about 9 to about 14.

The at least one alkaline material comprising at least one hydroxide-containing compound can be employed in the compositions of the present invention in an amount ranging from about 1 to about 30% by weight of active material, based on the total weight of the composition, including all ranges and subranges therebetween.

In certain embodiments, the at least one alkaline material comprising at least one hydroxide-containing compound is employed in the compositions of the present invention in an amount ranging from about 1 to about 20% by weight, preferably from about 1 to about 15% by weight, or more preferably from about 2 to about 15% by weight, or even more preferably from about 2 to about 10% by weight, such as from about 2 to about 6% by weight, with all weights of the alkaline material being the weight of the active material and based on the total weight of the composition, including all ranges and subranges therebetween.

In preferred embodiments, the at least one alkaline material comprising at least one hydroxide-containing compound is employed in an amount of about 10%, or about 8%, or about 6%, or about 5.5%, or about 5%, or about 4%, or about 3%, or about 2.8%, or about 2%, or about 1.5%, or about 1%, by weight, with all weights of the alkaline material being the weight of the active material and based on the total weight of the composition.

Crosslinked Copolymer of (Meth)Acrylic Acid and/or (C1-C6)Alkyl Esters

The at least one crosslinked copolymer of (meth)acrylic acid and/or (C1-C6)alkyl esters of the present invention is selected from a crosslinked (meth)acrylic acid/ethyl acrylate copolymer, a cross-linked anionic acrylate polymer, and mixtures thereof.

The crosslinked copolymer of (meth)acrylic acid and/or (C1-C6)alkyl esters of the present invention may also belong to a group of compounds known as acrylic thickening polymers.

The expression “thickening polymer” is understood, for the purposes of the present invention, to mean a polymer having, in solution or in dispersion containing 1% by weight of active material in water or in ethanol at 25° C., a viscosity greater than 0.2 poise at a shear rate of 1 s−1. The viscosity can be measured with a HAAKE RS600 viscometer from THERMO ELECTRON. This viscometer is a controlled-stress viscometer with cone-plate geometry (for example having a diameter of 60 mm).

As used herein, the term “(meth)acrylic” acid and “(meth)acrylate” are meant to include the corresponding methyl derivatives of acrylic acid and the corresponding alkyl acrylate. For example, “(meth)acrylic)” acid refers to acrylic acid and/or methacrylic acid and “(meth)acrylate” refers to alkyl acrylate and/or alkyl methacrylate.

In certain embodiments, the C1-C6 alkyl ester of the crosslinked copolymer of (meth)acrylic acid and/or (C1-C6)alkyl esters of the present invention is a C1-C6 alkyl acrylate.

Methacrylic acid is preferably present in amounts ranging from 20% to 80% by weight, more particularly from 25% to 70% by weight and even more particularly from 35% to 65% by weight relative to the total weight of the copolymer.

The alkyl acrylate is preferably present in amounts ranging from 15% to 80% by weight, more particularly from 25% to 75% by weight and even more particularly from 35% to 65% by weight relative to the total weight of the copolymer. It is chosen especially from methyl acrylate, ethyl acrylate and butyl acrylate and more particularly ethyl acrylate.

This copolymer is preferably partially or totally/substantially crosslinked with at least one standard polyethylenically unsaturated crosslinking agent, for instance polyalkenyl ethers of sucrose or of polyols, diallyl phthalates, divinylbenzene, allyl (meth)acrylate, ethylene glycol di(meth)acrylate, methylenebisacrylamide, trimethylolpropane tri(meth)acrylate, diallyl itaconate, diallyl fumarate, diallyl maleate, zinc (meth)acrylate, and castor oil or polyol derivatives manufactured from unsaturated carboxylic acids. The content of crosslinking agent generally ranges from 0.01% to 5% by weight, preferably from 0.03% to 3% by weight and even more particularly from 0.05% to 1% by weight relative to the total weight of the copolymer.

In preferred embodiments, the crosslinked copolymer of the present invention is slightly cross-linked.

As used herein, the term “slightly crosslinked” refers to a partially crosslinked three-dimensional polymeric network.

In other preferred embodiments, the crosslinked copolymer of the present invention is alkali-swellable.

As used herein, the term “alkali-swellable” as it pertains to the crosslinked copolymer of (meth)acrylic acid and/or (C1-C6)alkyl esters of the present invention refers to a polymer that when introduced to a solution, imparts little or no viscosity, but upon adjusting the pH to mildly acidic, neutral, or mildly basic conditions, a measurable increase in viscosity is observed, i.e., adding an alkali or neutralizing agent to a solution containing an alkali swellable polymer results in the development of viscosity.

The term “alkali-swellable” as used herein may also refer to the expansion of the polymer molecules upon neutralization as a result of charge repulsion of the anionic carboxylate groups of the polymer.

According to one particularly preferred form, the crosslinked copolymer of (meth)acrylic acid and/or (C1-C6)alkyl esters of the present invention selected from a crosslinked (meth)acrylic acid/ethyl acrylate copolymer and a cross-linked anionic acrylate polymer copolymer may especially be in the form of a dispersion in water. The mean size of the copolymer particles in the dispersion is generally between 10 and 500 nm, preferably between 20 and 200 nm and more preferentially from 50 to 150 nm.

In preferred embodiments, the crosslinked (meth)acrylic acid/ethyl acrylate copolymer is a crosslinked methacrylic acid/ethyl acrylate copolymer, also known as an acrylates copolymer contained in an aqueous dispersion, an example of which is a slightly cross-linked, alkali-swellable acrylate polymer known by the INCI name acrylates copolymer and commercially available from the supplier Lubrizol, under the tradename Carbopol® Aqua SF-1 as an aqueous dispersion comprising about 30% by weight of total solids or active material. In other words, the aqueous dispersion known under the tradename Carbopol® Aqua SF-1 comprises about 30% by weight of the acrylates copolymer, based on the weight of the dispersion.

Carbopol® Aqua SF-1 has a carboxyl functionality in its protonated form. It also has three structural units; one or more carboxylic acid monomers having 3 to 10 carbon atoms, one or more vinyl monomers and, one or more mono- or polyunsaturated monomers. It has an average 4.5 carbon alkyl side chains and more than 50% HM (the percentage hydrophobic modification (HM) of the polymer can be determined by the ratio of monomers added during synthesis, or by analytical techniques such as proton nuclear magnetic resonance (NMR)). This copolymer belongs to a class of synthetic rheology modifiers or thickener polymers that include carboxyl functional alkali-swellable and alkali-soluble thickeners (ASTs). These thickener polymers are prepared from the free-radical polymerization of acrylic acid alone or in combination with other ethylenically unsaturated monomers. The polymers can be synthesized by solvent/precipitation as well as emulsion polymerization techniques.

Other suitable crosslinked (meth)acrylic acid/ethyl acrylate copolymers may be chosen from a crosslinked copolymer of methacrylic acid and of ethyl acrylate as an aqueous dispersion containing 38% active material, commercially available from the company Coatex under the name Viscoatex™ 538C or a crosslinked copolymer of acrylic acid and of ethyl acrylate as an aqueous dispersion containing 28% active material, commercially available from the company Rohm and Haas and sold under the name Aculyn™ 33.

In other preferred embodiments, the crosslinked copolymer of (meth)acrylic acid and/or (C1-C6)alkyl esters of the present invention is a cross-linked anionic acrylate polymer. The cross-linked anionic acrylate polymer may be contained in an aqueous dispersion comprising about 32% by weight of total solids. Examples of the cross-linked anionic acrylate polymer of the present invention include, but are not limited to, the polymer known by the INCI name acrylates crosspolymer-4 and commercially available from the supplier Lubrizol, under the tradename Carbopol® Aqua SF-2, as an aqueous dispersion comprising about 32% by weight of total solids or active material. Acrylates Crosspolymer-4 may also be described as a copolymer of acrylic acid, methacrylic acid or one of its simple esters, crosslinked with trimethylolpropane triacrylate.

In certain embodiments of the present invention, the at least one crosslinked copolymer of (meth)acrylic acid and/or (C1-C6)alkyl esters comprises a crosslinked methacrylic acid/ethyl acrylate copolymer, preferably, an acrylates copolymer in aqueous dispersion, wherein the aqueous dispersion comprises about 30% by weight of total solids (acrylates copolymer). More preferably the acrylates copolymer in aqueous dispersion is a slightly cross-linked, alkali-swellable acrylate polymer known by the INCI name acrylates copolymer and commercially available from the supplier Lubrizol, under the tradename Carbopol® Aqua SF-1.

In other embodiments of the present invention, the at least one crosslinked copolymer of (meth)acrylic acid and/or (C1-C6)alkyl esters comprises a cross-linked anionic acrylate polymer, preferably contained in an aqueous dispersion comprising about 32% by weight of total solids and known by the INCI name acrylates crosspolymer-4 and commercially available from the supplier Lubrizol, under the tradename Carbopol® Aqua SF-2.

The at least one crosslinked copolymer of (meth)acrylic acid and/or (C1-C6)alkyl esters is employed in the composition of the present invention in an amount ranging from about 0.5 to about 15% by weight and preferably from about 1 to about 12% by weight, or preferably from about 1 to about 10% by weight, or preferably from about 1 to about 5% by weight, or more preferably, from about 1.95 to about 3.3% by weight, with all weights of the polymer being the weight of the active material and based on the total weight of the composition, including all ranges and subranges therebetween.

In some embodiments, the at least one crosslinked copolymer of (meth)acrylic acid and/or (C1-C6)alkyl esters is employed in the composition of the present invention in an amount of about 4%, or about 3.5%, or about 3.3%, or about 3%, or about 2.5%, or about 2.4%, or about 2%, or about 1.95% by weight, with all weights of the polymer being the weight of the active material and based on the total weight of the composition.

In other embodiments, when the at least one crosslinked copolymer of (meth)acrylic acid and/or (C1-C6)alkyl esters of the present invention is an acrylates copolymer in the form of an aqueous dispersion comprising about 30% by weight of total solids, the aqueous dispersion is employed in the compositions of the present invention in amount ranging from about 1 to about 15% by weight, preferably from about 2 to about 12% by weight, or more preferably from about 2.8 to about 11% by weight, or even more preferably from about 6.5 to about 11% by weight, based on the total weight of the composition, including all ranges and subranges therebetween.

In yet other embodiments, when the at least one crosslinked copolymer of (meth)acrylic acid and/or (C1-C6)alkyl esters of the present invention is an acrylates copolymer in the form of an aqueous dispersion comprising about 30% by weight of total solids, the aqueous dispersion is employed in the compositions of the present invention in amount of about 2.8% by weight, or such as about 6.5% by weight, or such as about 8% by weight, or such as about 11% by weight, based on the total weight of the composition.

### Neutralizing Agent

In preferred embodiments, the at least one crosslinked copolymer of (meth)acrylic acid and/or (C1-C6)alkyl esters of the present invention is neutralized in water or in an aqueous solution with a neutralizing agent before the polymer is added into the composition of the present invention.

In other preferred embodiments, the at least one crosslinked copolymer of (meth)acrylic acid and/or (C1-C6)alkyl esters of the present invention is neutralized with a neutralizing agent at the time of or after the addition of the polymer into the composition of the present invention.

The neutralizing agent is employed in an amount sufficient to neutralize the crosslinked copolymer of (meth)acrylic acid and/or (C1-C6)alkyl esters of the present invention in water or an aqueous solution. After neutralization, the crosslinked copolymer of (meth)acrylic acid and/or (C1-C6)alkyl esters may be partially or fully neutralized. One indication of neutralization is the clarity of the solution.

Suitable neutralizing agents may be selected from alkali metal carbonates, alkali metal phosphates, organic amines, hydroxide base compounds, and mixtures thereof, particularly from ethylamines, ethyleneamines, alkanolamines, cyclic amines and other cyclic compounds, saturated or unsaturated, having one or more nitrogen atoms within the ring.

The organic amines may be chosen from the ones having a pKb at 25° C. of less than 12, such as less than 10 or such as less than 6. It should be noted that this is the pKb corresponding to the function of highest basicity.

Organic amines may be chosen from organic amines comprising one or two primary, secondary, or tertiary amine functions, and at least one linear or branched C1-C8 alkyl groups bearing at least one hydroxyl radical.

Organic amines may also be chosen from alkanolamines such as mono-, di- or trialkanolamines, comprising one to three identical or different C1-C4 hydroxyalkyl radicals, ethylamines, ethyleneamines, quinoline, aniline and cyclic amines, such as pyrroline, pyrrole, pyrrolidine, imidazole, imidazolidine, imidazolidinine, morpholine, pyridine, piperidine, pyrimidine, piperazine, triazine and derivatives thereof.

Among the compounds of the alkanolamine type that may be mentioned include but not limited to: monoethanolamine (also known as monoethanolamine or MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, 2-amino-2-methyl-1-propanol, and tris(hydroxymethylamino)methane.

Other examples include but are not limited to: 1,3-diaminopropane, 1,3-diamino-2-propanol, spermine, and spermidine.

In some embodiments, the organic amines are chosen from amino acids.

As non-limiting examples, the amino acids that may be used may be of natural or synthetic origin, in L, D, or racemic form, and comprise at least one acid function chosen from, for instance, carboxylic acid, sulfonic acid, phosphonic acid, and phosphoric acid functions. The amino acids may be in their neutral or ionic form.

Amino acids that may be used in the present disclosure include but are not limited to: aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, and valine.

In some embodiments, the organic amines are chosen from organic amines of heterocyclic type, including but not limited to, pyridine, piperidine, imidazole, 1,2,4-triazole, tetrazole, and benzimidazole.

In some embodiments, the organic amines are chosen from amino acid dipeptides, including but not limited to: carnosine, anserine, and baleine.

In some embodiments, the organic amines are chosen from creatine, creatinine, 1,1-dimethylguanidine, 1,1-diethylguanidine, glycocyamine, metformin, agmatine, N-amidinoalanine, 3-guanidinopropionic acid, 4-guanidinobutyric acid, and 2-([amino(imino)methyl]amino)ethane-1-sulfonic acid.

The alkali metal phosphates and carbonates that may be used are, for example, sodium phosphate, potassium phosphate, sodium carbonate, sodium bicarbonate, potassium carbonate, potassium bicarbonate, and their derivatives.

The hydroxide base compounds that may be used include alkali metal hydroxides, alkaline-earth metal hydroxides, transition metal hydroxides, quaternary ammonium hydroxides, organic hydroxides, and mixtures thereof, such as those hydroxide-containing compounds comprising the alkaline material of the present invention.

According to at least one embodiment, the neutralizing agent is chosen from at least one organic amine such as at least one alkanolamine. Particularly preferred alkanolamines are ethanolamine (also known as monoethanolamine or MEA, triethanolamine or TEA, and 2-amino-2-methyl-1-propanol (also known as AMP), and mixtures thereof.

The at least one neutralizing agent of the present invention may be employed in an amount of from about 0.01% to about 5% by weight, or such as from about 0.1% to about 5% by weight, or such as from about 0.5% to about 3% by weight, based on the total weight of the cosmetic composition of the present invention.

In preferred embodiments, the at least one neutralizing agent of the present invention may be employed in an amount of from about 0.01% to about 2% by weight, or preferably from about 0.5% to about 1.5% by weight, or more preferably from about 0.6% to about 1.2% by weight, based on the total weight of the cosmetic composition of the present invention.

### Cosmetically Acceptable Solvent

The cosmetically acceptable solvent of the present invention is selected from water, organic solvents, and mixtures thereof. A water that is suitable for use in the invention may be a floral water such as cornflower water and/or a mineral water such as Vittel water, Lucas water or La Roche Posay water and/or a spring water and/or tap water and/or well water.

The organic solvent that may comprise the cosmetically acceptable solvent of the present is water-miscible (at room temperature: 25° C.). The water-miscible organic solvent can be chosen from monoalcohols containing from 2 to 6 carbon atoms, such as ethanol or isopropanol; polyols especially containing from 2 to 20 carbon atoms, preferably containing from 2 to 10 carbon atoms and preferentially containing from 2 to 6 carbon atoms, such as glycerol, propylene glycol, butylene glycol, pentylene glycol, hexylene glycol, dipropylene glycol or diethylene glycol; glycol ethers (especially containing from 3 to 16 carbon atoms) such as mono-, di- or tripropylene glycol (C1-C4)alkyl ethers, mono-, di- or triethylene glycol (C1-C4)alkyl ethers, and mixtures thereof.

In certain embodiments of the present invention, the organic solvent is chosen from propylene glycol, glycerin, and mixtures thereof.

The cosmetically acceptable solvent may also comprise stabilizers, for example sodium chloride, magnesium dichloride or magnesium sulfate.

The cosmetically acceptable solvent may also comprise any water-soluble or water-dispersible compound that is compatible with water or an aqueous phase, such as the above-described acrylic-containing polymers, thickening agents, liquid fatty substances, and mixtures thereof.

In some embodiments, the cosmetically acceptable solvent comprises 100% water by weight.

In particular, the cosmetically acceptable solvent of the invention may comprise water in a content ranging from about 45% to about 100% by weight, or from about 50% to about 90% by weight, or from about 50% to 80% by weight, based on the total weight of the composition, including all ranges and subranges there between.

### Surfactants

The compositions according to the invention may further comprise at least one surfactant selected from anionic surfactants, nonionic surfactants, amphoteric or zwitterionic surfactants and cationic surfactants, and mixtures thereof.

Anionic Surfactants

The term “anionic surfactant” is understood to mean a surfactant comprising, as ionic or ionizable groups, only anionic groups. These anionic groups are preferably chosen from the following groups: CO2H, CO2-, SO3H, SO3-, OSO3H, OSO3-, H2PO3, —HPO3-, —PO32-, —H2PO2, ═HPO2, —HPO2-, ═PO2-, ═POH and ═PO—.

Mention may be made, among the anionic surfactants capable of being used in the composition according to the invention, of alkyl sulfates, alkyl ether sulfates, alkylamido ether sulfates, alkylaryl polyether sulfates, monoglyceride sulfates, alkylsulfonates, alkylamidesulfonates, alkylarylsulfonates, α-olefinsulfonates, paraffinsulfonates, alkyl sulfosuccinates, alkyl ether sulfosuccinates, alkylamide sulfosuccinates, alkyl sulfoacetates, acyl sarcosinates, acyl glutamates, alykyl ether carboxylates, alkyl sulfosuccinamates, acyl isethionates and N-acyl taurates; monoalkyl esters of polyglycoside-polycarboxylic acids, acyl lactylates, salts of D-galactosideuronic acids, salts of alkyl ether carboxylic acids, salts of alkylaryl ether carboxylic acids, salts of alkylamido ether carboxylic acids; and the corresponding non-salified forms of all these compounds; the alkyl and acyl groups of all these compounds comprising from 6 to 24 carbon atoms and the aryl group denoting a phenyl group.

These compounds can be oxyethylenated and then preferably comprise from 1 to 50 ethylene oxide units and better still from 1 to 10 ethylene oxide units.

The salts of C6-C24 alkyl monoesters of polyglycoside-polycarboxylic acids can be chosen from C6-C24 alkyl polyglycoside-citrates, C6-C24 alkyl polyglycoside-tartrates and C6-C24 alkyl polyglycoside-sulfosuccinates.

The acyl lactylates preferably have an acyl group comprising from 8 to 20 carbon atoms.

When the anionic surfactant is in the salt form, it can be chosen from the alkali metal salts, such as the sodium salt or potassium salt, the ammonium salt, the amine salts and in particular the aminoalcohol salts, or the alkaline earth metal salts, such as the magnesium salt.

Use is preferably made of alkali metal or alkaline earth metal salts and in particular of sodium or magnesium salts.

The preferred anionic surfactants are chosen from (C6-24)alkyl sulfates, (C6-24)alkyl ether sulfates, acyl glutamates and (C6-C24)alkyl ether carboxylates, in particular in the form of alkali metal, ammonium, aminoalcohol or alkaline earth metal salts, or a mixture of these compounds.

In particular, use is preferably made of (C12-20)alkyl sulfates, (C12-20)alkyl ether sulfates comprising from 2 to 20 ethylene oxide units, acyl glutamates or (C12-C20)alkyl ether carboxylates, in particular in the form of alkali metal, ammonium, aminoalcohol and alkaline earth metal salts, or a mixture of these compounds.

### Nonionic Surfactants

In general, nonionic surfactants having a Hydrophilic-Lipophilic Balance (HLB) of from 8 to 20, are contemplated for use by the present invention. Examples of nonionic surfactants useful herein include, but are not limited to, alkoxylated derivatives of the following: fatty alcohols, alkyl phenols, fatty acids, fatty acid esters and fatty acid amides, wherein the alkyl chain is in the C12-050 range, preferably in the C16-C40 range, more preferably in the C24 to C40 range, and having from about 1 to about 110 alkoxy groups. The alkoxy groups are selected from the group consisting of C2-C6 oxides and their mixtures, with ethylene oxide, propylene oxide, and their mixtures being the preferred alkoxides. The alkyl chain may be linear, branched, saturated, or unsaturated. Of these alkoxylated non-ionic surfactants, the alkoxylated alcohols are preferred, and the ethoxylated alcohols and propoxylated alcohols are more preferred. The alkoxylated alcohols may be used alone or in mixtures thereof. The alkoxylated alcohols may also be used in mixtures with those alkoxylated materials disclosed herein-above.

Other representative examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), and steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Also available commercially are Brij® nonionic surfactants from Uniqema, Wilmington, Del. Typically, Brij® is the condensation products of aliphatic alcohols with from about 1 to about 54 moles of ethylene oxide, the alkyl chain of the alcohol being typically a linear chain and having from about 8 to about 22 carbon atoms, for example, Brij® 72 (i.e., Steareth-2) and Brij® 76 (i.e., Steareth-10).

Also useful herein as nonionic surfactants are alkyl glycosides, which are the condensation products of long chain alcohols, e.g. C8-C30 alcohols, with sugar or starch polymers. These compounds can be represented by the formula (S)n—O—R wherein S is a sugar moiety such as glucose, fructose, mannose, galactose, and the like; n is an integer of from about 1 to about 1000, and R is a C8-C30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkylpolyglucosides wherein S is a glucose moiety, R is a C8-C20 alkyl group, and n is an integer of from about 1 to about 9. Commercially available examples of these surfactants include decyl polyglucoside and lauryl polyglucoside, all the above-identified polyglucosides APG® are available from Cognis, Ambler, Pa. Also useful herein are sucrose ester surfactants such as sucrose cocoate and sucrose laurate.

Other nonionic surfactants suitable for use in the present invention are glyceryl esters and polyglyceryl esters, including but not limited to, glyceryl monoesters, preferably glyceryl monoesters of C16-C22 saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C16-C22 saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. Preferable are sorbitan esters of C16-C22 saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate, sorbitan sesquioleate, sorbitan monoisostearate, sorbitan stearates, sorbitan trioleate, sorbitan tristearate, sorbitan dipalmitates, and sorbitan isostearate. Sorbitan monoisostearate and sorbitan sesquioleate are particularly preferred emulsifiers for use in the present invention.

Also suitable for use herein are alkoxylated derivatives of glyceryl esters, sorbitan esters, and alkyl polyglycosides, wherein the alkoxy groups is selected from the group consisting of C2-C6 oxides and their mixtures, with ethoxylated or propoxylated derivatives of these materials being the preferred. Nonlimiting examples of commercially available ethoxylated materials include polysorbate materials sold under the tradename of TWEEN® by Croda, Inc. (ethoxylated sorbitan mono-, di- and/or tri-esters of C12 to C18 fatty acids with an average degree of ethoxylation of from about 2 to about 20).

Another suitable nonionic surfactant is an alkoxylated alcohol of glyceryl esters such as the polyethylene glycol derivative of hydrogenated castor oil, PEG-40 hydrogenated castor oil.

The at least one non-ionic surfactants may be chosen, for example, from polyethoxylated and/or polypropoxylated alkyl phenols, alpha-diols and alcohols, comprising fatty chains comprising, for example, from 8 to 18 carbon atoms, and the number of ethylene oxide and/or propylene oxide groups may range from 2 to 50. The at least one non-ionic surfactant may be chosen, for example, from copolymers of ethylene oxide and of propylene oxide, condensates of ethylene oxide and/or of propylene oxide with fatty alcohols or polyoxypropylene/polyoxyethylene ethers of fatty alcohol; polyethoxylated fatty amides comprising, for example, from 2 to 30 moles of ethylene oxide, polyglycerolated fatty amides comprising on average 1 to 5, and, for example, 1.5 to 4, glycerol groups; polyethoxylated fatty amines comprising, for example, from 2 to 30 moles of ethylene oxide, N-alkylglucamine derivatives, and amine oxides such as (C10-C14)alkyl amine oxides and N-acylaminopropylmorpholine oxides.

Suitable examples of nonionic surfactants for use in the invention are polysorbate compounds and derivatives such as polysorbate-21 and polysorbate-20, PEG-40 hydrogenated castor oil, PPG-5-Ceteth-20, alkylpolyglucosides such as caprylyl/capryl glucoside and decyl glucosides, and mixtures thereof.

In certain embodiments, when the compositions of the present invention contain at least one liquid fatty substance selected from mineral oil, the at least one nonionic surfactant is not selected from polysorbate compounds.

### Amphoteric or Zwitterionic Surfactants

The amphoteric or zwitterionic surfactants can in particular be derivatives of optionally quaternized secondary or tertiary aliphatic amines comprising at least one anionic group, such as, for exam-ple, a carboxylate, sulfonate, sulfate, phosphate or phosphonate group, and in which the aliphatic group or at least one of the aliphatic groups is a linear or branched chain comprising from 8 to 22 carbon atoms.

Mention may in particular be made of (C8-C20)alkyl betaines, sulfobetaines, (C8-C20)alkylamido(C1-C6)alkyl betaines, such as cocoamidopropyl betaine, or (C8-C20)alkylamido(C1-C6)alkyl sulfobetaines.

Mention may also be made of optionally quaternized secondary or tertiary aliphatic amines such as disodium cocoamphodiacetate, disodium lauroamphodiacetate, disodium caprylamphodiacetate, disodium caprylamphodiacetate, disodium cocoamphodi-propionate, disodium lauroamphodipropionate, disodium caprylamphodipropio-nate, disodium caprylamphodipropionate, lauroamphodipropionic acid and co-coampho [symbol] idipropionic acid.

Mention may be made, by way of example, of the cocoamphodiacetate sold by Rhodia under the trade name Miranol® C2M Concentrate.

Mention may also be made of the compound under the name sodium diethylaminopropyl cocoaspartamide and sold by Chimex under the name Chimexane HB.

Preferably, the amphoteric or zwitterionic surfactants are chosen from (C8-C20)alkyl betaines, (C8-C20)alkylamido(C1-C6)alkyl betaines and (C8-C20)alkylamphodiacetates, and also the sodium salt of diethylaminopropyl lauryla-minosuccinamate, and their mixtures.

Preferably, the amphoteric or zwitterionic surfactants are chosen, alone or as a mixture, from cocoylamidopropyl betaine (coco-betaine), cocoyl betaine and cocoamphodiacetate.

### Cationic Surfactants

The at least one cationic surfactant may be chosen, for example, from: salts of optionally polyoxyalkylenated primary, secondary and tertiary fatty amines; quaternary ammonium salts such as tetra alkyl ammonium, alkylamidoalkyltrialkyl ammonium, trialkylbenzyl ammonium, trialkylhydroxyalkyl ammonium and alkylpyridinium chlorides and bromides; imidazoline derivatives; and cationic amine oxides.

The at least one surfactant may be present in the composition and/or the aqueous composition in an amount ranging from about 0.01 to about 40%, such as from 0.05 to 30% by weight, or from about 0.1 to about 30% by weight, or from about 1 to about 20% by weight, or from about 1 to about 15% by weight, based on the total weight of the compositions.

### Liquid Fatty Substances

The compositions of the present invention may further comprise at least one liquid fatty substance, i.e. a compound that is liquid at a temperature of 25° C. and at atmospheric pressure (also called “oil”).

The term “fatty substance” means an organic compound that is insoluble in water at ordinary temperature (25° C.) and at atmospheric pressure (760 mmHg) (solubility of less than 5%, preferably less than 1% and even more preferentially less than 0.1%). They exhibit, in their structure, at least one hydrocarbon chain comprising at least 6 carbon atoms or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane. The fatty substances of the invention do not contain any salified or unsalified carboxylic acid groups (COOH or COO—).

The term “non-silicone o/V means an oil not containing any silicon atoms (Si) and the term “silicone o/V means an oil containing at least one silicon atom.

More particularly, the liquid fatty substances are chosen from C₆-C₁₆ hydrocarbons, hydrocarbons containing more than 16 carbon atoms, particularly linear or branched hydrocarbons of mineral or synthetic origin having more than 16 carbon atoms (e.g., mineral oil), non-silicone oils of animal origin, plant oils of triglyceride type, synthetic triglycerides, fluoro oils, liquid fatty alcohols, liquid fatty acid and/or liquid fatty alcohol esters other than triglycerides and plant waxes, silicones oils, and mixtures thereof.

The fatty alcohols, esters and acids more particularly have at least one linear or branched, saturated or unsaturated hydrocarbon-based group comprising 6 to 30 and better still from 8 to 30 carbon atoms, which is optionally substituted, in particular with one or more hydroxyl groups (in particular 1 to 4). If they are unsaturated, these compounds may comprise one to three conjugated or unconjugated carbon-carbon double bonds.

As regards the C₆-C₁₆ hydrocarbons, they are linear, branched or optionally cyclic, and are preferably alkanes. Examples that may be mentioned include hexane, dodecane, and isoparaffins such as isohexadecane, isododecane, and isodecane.

A hydrocarbon-based oil of animal origin that may be mentioned is perhydrosqualene.

The triglyceride oils of plant or synthetic origin are preferably chosen from liquid fatty acid triglycerides containing from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol® 810, 812 and 818 by the company Dynamit Nobel, jojoba oil and shea butter oil.

The linear or branched hydrocarbons of mineral or synthetic origin having more than 16 carbon atoms are preferably chosen from liquid paraffins, petroleum jelly, liquid petroleum jelly, polydecenes or hydrogenated polyisobutene, such as Parleam®. The fluoro oils that may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The liquid fatty alcohols which are suitable for the implementation of the invention are more particularly chosen from saturated or unsaturated, linear or branched alcohols comprising from 6 to 30 carbon atoms and preferably from 8 to 30 carbon atoms. Mention may be made, for example, of octyldodecanol, 2-butyloctanol, 2-hexyldecanol, 2-undecylpentadecanol, oleyl alcohol or linoleyl alcohol.

As regards the liquid fatty acids, mention may be made especially of saturated or unsaturated carboxylic acids comprising from 6 to 30 carbon atoms, and preferably from 9 to 30 carbon atoms, preferably chosen from oleic acid, linoleic acid, linolenic acid and isostearic acid. Theses acids are not under the form of salts, i.e. if present, the composition may not contain organic or mineral alkaline agents such as sodium hydroxide, potassium hydroxide, monoethanolamine, triethanolamine.

As regards the liquid esters of a fatty acid and/or of fatty alcohols, which are advantageously different from the triglycerides mentioned previously, mention may be made especially of liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total carbon number of the esters being greater than or equal to 6 and more advantageously greater than or equal to 10.

Among the monoesters, mention may be made of; isocetyl stearate; isodecyl neopentanoate; isostearyl neopentanoate; 2-ethylhexyl isononanoate; ethyl and isopropyl palmitates, alkyl myristates such as isopropyl, ethyl, myristate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may be made especially of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, it is preferred to use ethyl, isopropyl, myristyl, cetyl or stearyl palmitate, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The composition may also comprise, as liquid fatty ester, sugar esters and diesters of C₆-C₃₀ and preferably C₁₂-C₂₂ fatty acids. It is recalled that the term “sugar” means oxygen-bearing hydrocarbon-based compounds which have several alcohol functions, with or without aldehyde or ketone functions, and which comprise at least 4 carbon atoms. These sugars can be monosaccharides, oligosaccharides or polysaccharides.

Mention may be made, as suitable sugars, for example, of sucrose (or saccharose), glucose, galactose, ribose, fucose, maltose, fructose, mannose, arabinose, xylose and lactose, and derivatives thereof, in particular alkyl derivatives, such as methyl derivatives, for instance methylglucose.

The sugar esters of fatty acids may be chosen in particular from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters can, for example, be oleates, laurates, palmitates, myristates, behenates, cocoates, stearates, linoleates, linolenates, caprates, arachidonates or mixtures thereof, such as, in particular, oleate/palmitate, oleate/stearate or palmitate/stearate mixed esters.

More particularly, use is made of monoesters and diesters and in particular mono- or di-oleate, -stearate, -behenate, -oleate/palmitate, -linoleate, -linolenate or—oleate/stearate of sucrose, of glucose or of methylglucose.

An example that may be mentioned is the product sold under the name Glucate® DO by the company Amerchol, which is a methylglucose dioleate.

The silicones oils that may be used in the composition of the present invention are volatile or non-volatile, cyclic, linear or branched silicones, which are unmodified or modified with organic groups, having a viscosity from 5×10⁻6 to 2.5 m²/s at 25° C., and preferably 1×10⁻5 to 1 m²/s.

Preferably, the silicone is chosen from liquid polydialkylsiloxanes, especially polydimethylsiloxanes (PDMS), and liquid organomodified polysiloxanes comprising at least one functional group chosen from amino groups and alkoxy groups.

Organopolysiloxanes are defined in greater detail in Walter Noll's Chemistry and Technology of Silicones (1968), Academic Press. They may be volatile or nonvolatile.

When they are volatile, the silicones are more particularly chosen from those having a boiling point of between 60° C. and 260° C., and more particularly still from:
(i) cyclic polydialkylsiloxanes containing from 3 to 7 and preferably from 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, and Silbione® 70045 V5 by Rhodia, and mixtures thereof.
Mention may also be made of cyclocopolymers of the dimethylsiloxane/methylalkylsiloxane type, such as Volatile Silicone® FZ 3109 sold by the company Union Carbide.
Mention may also be made of mixtures of cyclic polydialkylsiloxanes with organosilicon compounds, such as the mixture of octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane;
(ii) linear volatile polydialkylsiloxanes containing 2 to 9 silicon atoms and having a viscosity of less than or equal to 5×10⁻6 m2/s at 25° C. An example is decamethyltetrasiloxane sold in particular under the name SH 200 by the company Toray Silicone.

Use may be made of non-volatile polydialkylsiloxanes, among which mention may be made mainly of polydimethylsiloxanes having trimethylsilyl end groups. The viscosity of the silicones is measured at 25° C. according to ASTM standard 445 Appendix C.

Mention may be made, among these polydialkylsiloxanes, without implied limitation, of the following commercial products:
the Silbione® oils of the 47 and 70 047 series or the Mirasil® oils sold by Rhodia, such as, for example, the oil 70 047 V 500 000;
the oils of the Mirasil® series sold by Rhodia;
the oils of the 200 series from the company Dow Corning, such as DC200 with a viscosity of 60 000 mm2/s;
the Viscasil® oils from General Electric and certain oils of the SF series (SF 96, SF 18) from General Electric.

Mention may also be made of polydimethylsiloxanes bearing dimethylsilanol end groups known under the name dimethiconol (CTFA), such as the oils of series 48 from the company Rhodia.

The liquid fatty substances are advantageously chosen from alkanes, non-silicone oils of plant, mineral or synthetic origin, liquid fatty alcohols, liquid fatty acids and liquid esters of a fatty acid and/or of a fatty alcohol, or mixtures thereof.

Preferably, the liquid fatty substance is chosen from liquid petroleum jelly, C₆-C₁₆ alkanes, mineral oil, polydecenes, liquid esters of a fatty acid and/or of a fatty alcohol, and liquid fatty alcohols, or mixtures thereof.

A preferred liquid fatty substance for use in the present invention is mineral oil which may be commercially available from the supplier Sonneborn under the tradename Kaydol® Heavy White Mineral Oil or from the supplier Exxonmobil Chemical under the tradename Primol™ 352 or from Sonneborn under the tradename Blandol, or from Armedsa under the tradename Aemoil M-302CG or from Exxonmobil Chemical under the tradename Marcol 82.

The at least one liquid fatty substance may be employed in the composition of the present invention in an amount of at least about 10% by weight, based on the total weight of the composition.

In some embodiments, the at least one liquid fatty substance is employed in the composition of the present invention in an amount of at least about 10%, or at least about 20% by weight, or at least about 10% by weight, or at least about 20% by weight, or at least about 30% by weight, or at least about 40% by weight, based on the total weight of the composition.

In certain embodiments, the at least one liquid fatty substance is present in the composition of the present invention in an amount ranging from about 10 to about 60% by weight, preferably in an amount ranging from about 20 to about 60% by weight, or more preferably in an amount ranging from about 30 to about 60% by weight, or even more preferably in an amount ranging from about 40 to about 60% by weight, based on the total weight of the composition, including all ranges and subranges therebetween.

In yet other embodiments, the at least one liquid fatty substance may be employed in an amount of about 10%, or about 20%, or about 30%, or about 40%, or about 50%, or about 60% by weight, based on the total weight of the composition.

### Thickening Agents

The compositions according to various embodiments of the disclosure can further comprise at least one component chosen from thickening agents, also referred to interchangeably herein as thickeners or rheology modifiers/rheology modifying agents. Thickening agents are generally used to modify the viscosity or rheology of compositions. Thickening agents that may further comprise the compositions of the present invention are those other than the crosslinked copolymer of (meth)acrylic acid and/or (C1-C6)alkyl esters selected from a crosslinked (meth)acrylic acid/ethyl acrylate copolymer and a cross-linked anionic acrylate polymer as described above.

Non-limiting examples of thickening agents that may be used according to various embodiments of the disclosure include those conventionally used in cosmetics, such as polymers of natural origin and synthetic polymers. For example, nonionic, anionic, cationic, amphiphilic, and amphoteric polymers, and other known rheology modifiers, such as cellulose-based thickeners, may be chosen.

The thickening agents may be chosen from, for example, hydrophilic thickeners, for example cellulose polymers and gums. As used herein, the term “hydrophilic thickener” is meant to indicate that the thickening agent is soluble or dispersible in water. Non-limiting examples of hydrophilic thickeners include modified or unmodified carboxyvinyl polymers, such as the products sold under the name CARBOPOL (CTFA name: carbomer) by Goodrich, homopolymers or copolymers of acrylic or methacrylic acids or the salts thereof and the esters thereof, such as the products sold under the names VERSICOL F® or VERSICOL K® by Allied Colloid, ULTRAHOLD 8® by Ciba-Geigy, polyacrylates and polymethacrylates such as the products sold under the names LUBRAJEL and NORGEL by Guardian, or under the name HISPAJEL by Hispano Chimica, and polyacrylic acids of SYNTHALEN K type, polyacrylamides, copolymers of acrylic acid and of acrylamide sold in the form of the sodium salt thereof, such as under the names RETEN® by Hercules, the sodium polymethacrylate such as sold under the name DARVAN 7® by Vanderbilt, and the sodium salts of polyhydroxycarboxylic acids such as sold under the name HYDAGEN F® by Henkel, optionally crosslinked and/or neutralized 2-acrylamido-2-methylpropanesulphonic acid polymers and copolymers, for instance poly(2-acrylamido-2-methylpropanesulphonic acid) such as sold by Clariant under the name HOSTACERIN AMPS (CTFA name: ammonium polyacryldimethyltauramide), crosslinked anionic copolymers of acrylamide and of AMPS, e.g. in the form of a water-in-oil emulsion, such as those sold under the name SEPIGEL™ 305 (CTFA name: Polyacrylamide/C13-14 lsoparaffin/Laureth-7) and under the name SIMULGEL™ 600 (CTFA name: Acrylamide/Sodium acryloyldimethyltaurate copolymer/Isohexadecane/Polysorbate 80) by SEPPIC, polyacrylic acid/alkyl acrylate copolymers of PEMULEN type, associative polymers, for instance PEG-150/stearyl alcohol/SMDI copolymer such as sold under the name ACULYN™ 46 by Rohm & Haas, steareth-100/PEG-136/HDI copolymer such as sold under the name RHEOLATE® FX 1100 by Elementis), as well as mixtures thereof.

Other exemplary hydrophilic thickeners include associative polymers. As used herein, the term “associative polymer” is intended to mean any amphiphilic polymer comprising in its structure at least one fatty chain and at least one hydrophilic portion. The associative polymers in accordance various exemplary embodiments may be anionic, cationic, nonionic or amphoteric. By way of example, associative polymers which may be chosen include those comprising at least one hydrophilic unit and at least one fatty-chain allyl ether unit, such as those in which the hydrophilic unit is constituted of an ethylenic unsaturated anionic monomer, such as a vinylcarboxylic acid or an acrylic acid, a methacrylic acid, and mixtures thereof, and in which the fatty-chain allyl ether unit corresponds to the monomer of formula (I) below:

CH₂═C(R′)CH₂OBₙR (I)

in which R′ is chosen from H or CH₃, B is chosen from an ethyleneoxy radical, n is zero or is chosen from an integer ranging from 1 to 100, and R is chosen from a hydrocarbon-based radical chosen from alkyl, arylalkyl, aryl, alkylaryl and cycloalkyl radicals containing from 8 to 30 carbon atoms, such as from 10 to 24 carbon atoms, or from 12 to 18 carbon atoms.

Non-limiting examples of associative anionic polymers that may also be chosen include anionic polymers comprising at least one hydrophilic unit of olefinic unsaturated carboxylic acid type, and at least one hydrophobic unit exclusively of (C₁₀-C₃₀)alkyl ester of unsaturated carboxylic acid type.

In certain exemplary and non-limiting embodiments, the thickening copolymers are chosen from the copolymers resulting from the polymerization of:
(1) at least one monomer of formula (II):

CH₂═CH(R₁)COOH (II)
wherein R₁ is chosen from H or CH₃ or C₂H₅, providing acrylic acid, methacrylic acid, or ethacrylic acid monomers, and
(2) at least one monomer of (C₁₀-C₃₀)alkyl ester of unsaturated carboxylic acid type corresponding to the monomer of formula (III):

CH₂═CH(R₂)COOR₃ (III)

wherein R₂ is chosen from H or CH₃ or C₂H₅, providing acrylate, methacrylate or ethacrylate units, R₃ denoting a C₁₀-C₃₀ alkyl radical, such as a C₁₂-C₂₂ alkyl radical.

Non-limiting examples of (C₁₀-C₃₀)alkyl esters of unsaturated carboxylic acids are for example chosen from lauryl acrylate, stearyl acrylate, decyl acrylate, isodecyl acrylate, dodecyl acrylate and the corresponding methacrylates, such as lauryl methacrylate, stearyl methacrylate, decyl methacrylate, isodecyl methacrylate and dodecyl methacrylate, and mixtures thereof.

Additionally, crosslinked thickening polymers may be chosen according to further exemplary embodiments. For example, such polymers may be chosen from polymers resulting from the polymerization of a mixture of monomers comprising:

(1) acrylic acid,
(2) an ester of formula (III) described above, in which R₂ is chosen from H or CH₃, R₃ denoting an alkyl radical having from 12 to 22 carbon atoms, and
(3) a crosslinking agent, which is a well-known copolymerizable polyethylenic unsaturated monomer, such as diallyl phthalate, allyl (meth)acrylate, divinylbenzene, (poly)ethylene glycol dimethacrylate and methylenebisacrylamide.

By way of example, crosslinked thickening polymers comprising about 60% to about 95% by weight of acrylic acid (hydrophilic unit), about 4% to about 40% by weight of C₁₀-C₃₀ alkyl acrylate (hydrophobic unit), and about 0% to about 6% by weight of crosslinking polymerizable monomer. In yet further embodiments, the crosslinked thickening polymers may comprise about 96% to about 98% by weight of acrylic acid (hydrophilic unit), about 1% to about 4% by weight of C₁₀-C₃₀ alkyl acrylate (hydrophobic unit), and about 0.1% to 0.6% by weight of crosslinking polymerizable monomer, such as those described above.

For example, acrylate/C₁₀-C₃₀ alkyl acrylate copolymers (INCI name: Acrylates/C10-30 Alkyl Acrylate Crosspolymer), such as the products sold by Lubrizol under the trade names PEMULEN™ TR1, PEMULEN™ TR2, CARBOPOL® 1382 and CARBOPOL® EDT 2020 may be chosen.

Other thickening agents that may be optionally be used in the invention are crosslinked copolymers comprising from 2.5% to 65% (meth)acrylic acid residues, from 10% to 80% C2-C4 alkyl (meth)acrylate residues, from 2% to 25% lipophilically modified (meth)acrylate residues and residues of a crosslinker that has no ester or amide functionality.

Cationic associative polymers that may be chosen include, but are not limited to, quaternized cellulose derivatives and polyacrylates containing amine side groups.

Exemplary non-ionic associative polymers include celluloses modified with groups comprising at least one fatty chain, for instance hydroxyethyl celluloses modified with groups comprising at least one fatty chain, such as alkyl groups, e.g. C₈-C₂₂ alkyl groups, arylalkyl and alkylaryl groups, such as cetyl hydroxyethyl cellulose, also known as Natrosol® Plus (sold by the company Ashland); Bermocoll EHM 100 (sold by the company Berol Nobel), Amercell Polymer HM-1500® sold by Amerchol (hydroxyethylcellulose modified with a polyethylene glycol (15) nonylphenyl ether group, sold by the company Amerchol), celluloses modified with polyalkylene glycol alkylphenyl ether groups, guars such as hydroxypropyl guar, optionally modified with groups comprising at least one fatty chain such as an alkyl chain, for example JAGUAR® XC-95/3 (C14 alkyl chain, sold by the company Rhodia Chimie); Esaflor HM 22 (C22 alkyl chain, sold by the company Lamberti); RE210-18 (C14 alkyl chain) and RE205-1 (C20 alkyl chain, sold by the company Rhodia Chimie), copolymers of vinylpyrrolidone and of fatty-chain hydrophobic monomers, for instance Antaron® or Ganex® V216 (vinylpyrrolidone/hexadecene copolymers); Antaron® or Ganex® V220 (vinylpyrrolidone/eicosene copolymers), sold by the company I.S.P., copolymers of C₁-C₆ alkyl methacrylates or acrylates and of amphiphilic monomers comprising at least one fatty chain, and copolymers of hydrophilic methacrylates or acrylates and of hydrophobic monomers comprising at least one fatty chain, for instance the polyethylene glycol methacrylate/lauryl methacrylate copolymer; polymers with an aminoplast ether skeleton containing at least one fatty chain, such as the Pure Thix® nonionic associative water phase thickeners sold by the company Southern Clay Products, Inc.

Associative polyurethanes may also be chosen in various exemplary and non-limiting embodiments. These are nonionic block copolymers comprising in the chain both hydrophilic blocks usually of polyoxyethylene nature, and hydrophobic blocks that may be aliphatic sequences alone and/or cycloaliphatic and/or aromatic sequences. Associative polyurethanes comprise at least two hydrocarbon-based lipophilic chains containing from C₆ to C₃₀ carbon atoms, separated by a hydrophilic block, the hydrocarbon-based chains optionally being pendent chains or chains at the end of a hydrophilic block. For example, it is possible for one or more pendent chains to be provided. In addition, the polymer may comprise a hydrocarbon-based chain at one or both ends of a hydrophilic block. The associative polyurethanes may be arranged in triblock or multiblock form. The hydrophobic blocks may thus be at the each end of the chain (for example, triblock copolymer with a hydrophilic central block) or distributed both at the ends and within the chain (for example, multiblock copolymer). These polymers may also be graft polymers or starburst polymers. For example, the associative polyurethanes may be triblock copolymers in which the hydrophilic block is a polyoxyethylene chain containing from 50 to 1000 oxyethylene groups.

By way of non-limiting example, associative polymers of the polyurethane polyether type that may be used include the polymer C₁₆-0E₁₂₀-C₁₆ from Servo Delden (under the name SER AD FX1100), which is a molecule containing a urethane function and having a weight-average molecular weight of 1300), OE being an oxyethylene unit, Nuvis® FX 1100 (European and US INCI name “Steareth-100/PEG-136/HMDI Copolymer” sold by the company Elementis Specialties), and also Acrysol RM 184® (sold by the company Rohm and Haas); Elfacos® T210® (C12-C14 alkyl chain) and Elfacos® T212® (C18 alkyl chain) sold by the company Akzo. Further exemplary associative polymers that may be chosen include RHEOLATE® 205 containing a urea function, sold by Rheox, or RHEOLATE® 208 or 204, or RHEOLATE® FX1100 from Elementis. The product DW 1206B from Rohm & Haas containing a C₂₀ alkyl chain with a urethane bond, sold at a solids content of 20% in water, may also be used.

In yet further exemplary embodiments, solutions or dispersions of these polymers, especially in water or in aqueous-alcoholic medium, may be chosen. In at least one exemplary embodiment, the at least one thickening agent is chosen from copolymers resulting from the polymerization of at least one monomer (a) chosen from carboxylic acids possessing α,β-ethylenically unsaturated groups or their esters, with at least one monomer (b) possessing ethylenically unsaturated groups and comprising a hydrophobic group. Such copolymers may exhibit emulsifying properties.

As used herein, the term “copolymers” is intended to mean both copolymers obtained from two types of monomers and those obtained from more than two types of monomers, such as, for example, terpolymers obtained from three types of monomers. The chemical structure of the copolymers comprises at least one hydrophilic unit and at least one hydrophobic unit. The expression “hydrophobic unit” or “hydrophobic unit” is understood to mean a radical possessing a saturated or unsaturated and linear or branched hydrocarbon-based chain which comprises at least 8 carbon atoms, for example from 10 to 30 carbon atoms, as a further example from 12 to 30 carbon atoms, and as yet a further example from 18 to 30 carbon atoms.

In further embodiments, the at least one thickening agent may be chosen from nonionic homopolymers or copolymers containing ethylenically unsaturated monomers of the ester and/or amide type.

In yet further embodiments, the at least one thickening agent chosen from polymers of natural origin may include, for example, thickening polymers comprising at least one sugar unit, for instance nonionic guar gums, optionally modified with C1-C6 hydroxyalkyl groups; biopolysaccharide gums of microbial origin, such as scleroglucan gum (also known as sclerotium gum) or xanthan gum; gums derived from plant exudates, such as gum arabic, ghatti gum, karaya gum, gum tragacanth, carrageenan gum, agar gum and carob gum, ceratonia siliqua gum and cyamopsis tetragonoloba (guar) gum; pectins; alginates; starches; hydroxy(C1-C6)alkylcelluloses and carboxy(C1-C6)alkylcelluloses.

Non-limiting examples of nonionic, unmodified guar gums that may be used in various embodiments include Guargel D/15 (Noveon); Vidogum GH 175 (Unipectine), Meypro-Guar 50 and JAGUAR® C (Meyhall/Rhodia Chimie). Non-limiting examples of nonionic modified guar gums include Jaguar® HP8, HP60, HP120, DC 293 and HP 105 (Meyhall/Rhodia Chimie); and Galactasol 4H4FD2 (Ashland).

Further examples of useful thickening agents include scleroglucans, for example, Actigum™ CS from Sanofi Bio Industries; Amigel from Alban Muller International, and also the glyoxal-treated scleroglucans described in FR2633940); xanthan gums, for instance Keltrol®, Keltrol® T, Keltrol® Tf, Keltrol® Bt, Keltrol® Rd, Keltrol® Cg (Nutrasweet Kelco), Rhodicare® S and Rhodicare® H (Rhodia Chimie); starch derivatives, for instance Primogel® (Avebe); hydroxyethylcelluloses such as Cellosize® QP3L, QP4400H, QP30000H, HEC30000A and Polymer PCG10 (Amerchol), Natrosol™ 250HHR®, 250MR, 250M, 250HHXR, 250HHX, 250HR, HX (Hercules) and Tylose® H1000 (Hoechst); hydroxypropylcelluloses, for instance Klucel® EF, H, LHF, MF and G (Ashland); carboxymethylcelluloses, for instance Blanose® 7M8/SF, refined 7M, 7LF, 7MF, 9M31F, 12M31XP, 12M31P, 9M31XF, 7H, 7M31, 7H3SXF (Ashland), Aquasorb® A500 (Hercules), Ambergum® 1221 (Hercules), Cellogen® HP810A, HP6HS9 (Montello) and Primellose® (Avebe).

Exemplary modified nonionic guar gums may, for example, be modified with C1-C6 hydroxyalkyl groups. Exemplary hydroxyalkyl groups may include hydroxymethyl, hydroxyethyl, hydroxypropyl and hydroxybutyl groups.

Guar gums are well known in the state of the art and may, for example, be prepared by reacting the corresponding alkene oxides, such as for example propylene oxides, with guar gum so as to obtain a guar gum modified with hydroxypropyl groups. The hydroxyalkylation ratio, which corresponds to the number of alkylene oxide molecules consumed to the number of free hydroxyl functional groups present on the guar gum, may in at least certain exemplary embodiments vary from about 0.4 to about 1.2.

Exemplary and non-limiting nonionic guar gums, optionally modified with hydroxyalkyl groups, include those sold under the trade names JAGUAR® HP8, JAGUAR® HP60 and JAGUAR® HP120, JAGUAR® DC 293 and JAGUAR® HP 105 by the company RHODIA CHIMIE (RHODIA CHIMIE), and under the name GALACTASOL™ 4H4FD2 by the company ASHLAND.

Guar gums may also be modified with a quaternary ammonium group. Guar gums modified as such include Guar Hydroxypropyltrimonium Chloride, also known under the tradename JAGUAR® C-13S (RHODIA CHIMIE).

Exemplary and non-limiting celluloses include hydroxyethylcelluloses and hydroxypropylcelluloses. The products sold under the names KLUCEL EF, KLUCEL H, KLUCEL LHF, KLUCEL MF, KLUCEL G, by the company ASHLAND, CELLOSIZE POLYMER PCG-10 by the company AMERCHOL, may be chosen in various embodiments.

Exemplary, non-limiting thickening polysaccharides may be chosen from glucans, modified or unmodified starches (such as those derived, for example, from cereals such as wheat, corn or rice, vegetables such as golden pea, tubers such as potato or cassava), amylose, amylopectin, glycogen, dextrans, celluloses and derivatives thereof (methylcelluloses, hydroxyalkylcelluloses, ethylhydroxyethylcelluloses), mannans, xylans, lignins, arabans, galactans, galacturonans, chitin, chitosans, glucoronoxylans, arabinoxylans, xyloglucans, glucomannans, pectic acids and pectins, arabinogalactans, carrageenans, agars, gums arabic, gums tragacanth, Ghatti gums, Karaya gums, carob gums, galactomannans such as guar gums and their nonionic derivatives (hydroxypropylguar), and mixtures thereof.\Further, exemplary thickening agents include silicas, optionally hydrophobic, such as those described in EP-A-898960, and for example marketed as AEROSIL® R812 by the company Degussa, CAB-O-SIL TS-530, CAB-O-SIL TS-610, CAB-O-SIL TS-720 by the company Cabot, AEROSIL® R972, AEROSIL® R974 by the company Degussa; clays, such as montmorillonite, modified clays such as the bentones for example, stearalkonium hectorite, stearalkonium bentonite; polysaccharide alkyl ethers (optionally with the alkyl group having from 1 to 24 carbon atoms, for example from 1 to 10 carbon atoms, as a further example from 1 to 6 carbon atoms, and as yet a further example from 1 to 3 carbon atoms) such as those described in document EP-A-898958.\Thickening agents of the present disclosure may also include rheology modifiers. In accordance with the disclosure, rheology modifiers may, in various exemplary embodiments, be chosen from Polyacrylamide(and)C13-14 Isoparaffin(and)Laureth-7 (Sepigel™ 305 from Seppic), Hydroxypropyl Guar (JAGUAR® HP105 from Rhodia), Cyamopsis Tetragonoloba (Guar) Gum (Supercol U Guar Gum from Ashland), Acrylates/C10-30 Alkyl Acrylate Crosspolymer (Carbopol® Ultrez 20 Polymer from Lubrizol), Acrylates/C10-30 Alkyl Acrylate Crosspolymer (Permulen™ TR-1 from Lubrizol), Polyacrylate Crosspolymer-6 (Sepimax Zen from Seppic), Sclerotium Gum (Amigum from Alban Muller), Xanthan Gum(and)Ceratonia Siliqua Gum (Nomcort CG from Nisshin Oil Lio), Hydroxypropyl Guar (Jaguar® HP8 from Rhodia), Guar Hydroxypropyl Trimonium Chloride (Jaguar® C-13-S from Rhodia), Hydroxyethyl Cellulose (Natrosol® 250 MR from Ashland). When anionic thickening agents are used, they are generally neutralized before being included in or as they are added to the compositions of the disclosure.

Cationic thickening agents of the disclosure may also be chosen from non-associative cationic polymers such as dimethylaminoethyl methacrylate homopolymers quaternized with methyl chloride or dimethylaminoethyl methacrylate copolymers quaternized with methyl chloride and acrylamide. Among the homopolymers of this type, mention may be made of the products sold under the names Salcare SC95 and Salcare SC96 by the company Ciba and SYNTHALEN® CR by the company 3V Sigma (chemical name: methacryloylethyl trimethyl ammonium chloride homopolymer, INCI name: polyquaternium-37). Among the copolymers of this family, mention may be made of the product Salcare S C92 sold by Ciba or the product PAS 5 194 sold by Hoechst.

Another suitable example of a cationic thickening agent is a product known by the INCI name of polyacrylate-1 crosspolymer (Carbopol® Aqua CC, from the company, Lubrizol).

It is contemplated that, in at least certain exemplary and non-limiting embodiments, the thickening agents of the disclosure may include compounds such as gellifying and viscosity modifying agents. For example, compositions of the disclosure may employ at least one water-soluble resin such as polyethylene oxide having a molecular weight ranging from 100,000 to Ser. No. 10/000,000. Examples of such polyethylene oxides include, but not limited to, Polyox water-soluble resins manufactured by Dow under the INCI names of PEG-2M, PEG-5M, PEG-7M, PEG-14M, PEG-23M, PEG-45M, PEG-90M, PEG-160M, and PEG-180M. PEG-90M is known under the tradename of Polyox™ WSR 301, and PEG-45M is known under the tradename Polyox™ WSR 60k.

The thickening agents of the present invention also includes silica material, starches, and clay.

The silica material may comprise silica particles that can be hydrophilic or hydrophobic silicas or mixtures thereof.

The starch that can be used in the present invention may originate from a plant source such as cereals, tubers, roots, legumes and fruit. Thus, the starch(es) may originate from a plant source chosen from corn, pea, potato, sweet potato, banana, barley, wheat, rice, oat, sago, tapioca and sorghum.

The clays of the present invention may be chosen from clays of the family of the smectites, such as laponite and montmorillonite, of the family of the kaolinites, such as kaolinite, dickite or nacrite, optionally modified clays of the family of halloysite, donbassite, antigorite, berthierine or pyrophyllite, montmorillonites, beidellite, vermiculites, talc, stevensite, hectorites, bentonites, saponites, chlorites, sepiolite and illite.

The clay or clays of the present invention can be natural or synthetic. Natural clay is a sedimentary rock in large part composed of specific minerals, silicates, generally, of aluminum. Kaolin is thus a natural clay.

Clays can also be chemically modified by various compounds, such as acrylic acids, polysaccharides (for example carboxymethylcellulose) or organic cations.

Use is preferably made, in the context of the present invention, of clays which are cosmetically compatible with and acceptable to the hair, skin and/or scalp.

The amounts of thickening agents in the compositions of the disclosure may range from about 0.1% to about 20% by weight, or such as from about 0.5% to about 15% by weight, or such as from about 0.5% to about 10% by weight, or such as from about 1% to about 5% by weight, relative to the total weight of the composition.

### Auxiliary Ingredients

The compositions according to the invention may also comprise any auxiliary ingredient usually used in the field under consideration, selected, for example, from emulsifying agents, conditioning agents, moisturizing agents, shine agents, fillers, colorants, pigments, chelating compounds, sequestering agents, fragrances, preservatives, stabilizers, and mixtures thereof.

It is a matter of routine operations for a person skilled in the art to adjust the nature and amount of the additives present in the compositions in accordance with the invention such that the desired cosmetic properties and stability properties thereof are not thereby affected.

Emulsifying agents of the present invention may be chosen from the nonionic surfactants as described above.

The emulsifying agents may also be chosen from polyether-modified silicones having a long chain of dimethyl siloxane units which carry polyethoxy-polypropoxy units in the chain and at the ends. Examples include cyclopentasiloxane PEG/PPG-18/18 dimethicone, PEG-12 Dimethicone, and PEG/PPG-19/19 Dimethicone.

Conditioning agents of the present invention may be chosen from polymeric and non-polymeric cationic compounds. The earlier-described cationic surfactants may also be used as conditioning agents in the compositions of the present invention.

Suitable examples of conditioning agents may be chosen from allantoin, cationic compounds such as polyquaternium-39, behentrimonium chloride, cetrimonium chloride, polyquaternium-22, polyquaternium-6, polyquaternium-10, behentrimonium methosulfate, and mixtures thereof.

Colorants of the present invention may be chosen from non-reactive dyes, temporary dyes, natural dyes, and mixtures thereof. When the composition of the present invention is clear in appearance, a colorant may be included in order to impart a tint or color to the composition.

The chelating compounds of the present invention may be chosen from ethylene diamine tetraacetic acid (EDTA) and its salts; N-(hydroxyethyl) ethylene diamine triacetic acid and its salts; aminotrimethylene phosphonic acid and its salts; diethylenetriamine-pentaacetatic acid and its salts; lauroyl ethylene diamine triacetic acid and its salts; nitrilotriacetic acid and its salts; iminodisuccinic acid and its salts; tartaric acid and its salts; citric acid and its salts; N-2-hydroxyethyliminodiacetic acid and its salts; ethyleneglycol-bis(beta-amino ethyl ether)-N,N-tetraacetic acid; and pentasodium aminotrimethylene phosphonate. The salts may be chosen from salts with organic or inorganic cations. In one embodiment, the inorganic cation is chosen from potassium, sodium or lithium.

In a further preferred embodiment, a salt of EDTA, such as sodium, lithium, potassium or guanidine EDTA, is the complexing agent.

The at least one chelating compound may also be combined with at least one sequestering agent.

The at least one chelating compound may be present in the compositions of the invention in an amount of from about 0.5% to about 5% by weight, preferably from about 0.8% to about 4% by weight, more preferably from about 0.8% to about 3% by weight, and more preferably from about 1% to about 2.5% by weight, such as at about 1%, or about 1.5%, or about 2%, or about 2.5%, based on the total weight of the composition.

### Compositions

The present invention relates to a composition comprising an alkaline material comprising hydroxide-containing compounds, a crosslinked copolymer of (meth)acrylic acid and/or (C1-C6)alkyl esters which is preferably neutralized, and a cosmetically acceptable solvent. Said composition can additionally contain a liquid fatty substance, a thickening agent, a surfactant, and an auxiliary ingredient.

The compositions of the present invention may be in the form of a liquid, a gel, a lotion, or a cream. In certain embodiments, the composition of the present invention can be an aqueous gel, lotion or solution.

In some preferred embodiments, the compositions of the present invention are in the form of aqueous gels that are clear in appearance.

In other embodiments, when the composition of the present invention contains at least one of the above-described liquid fatty substances in an amount of at least about 10% by weight, based on the total weight of the composition, the composition of the present invention is the form of a emulsion, preferably, an oil-in-water emulsion. Thus, in certain embodiments, the at least one liquid fatty substances is present in the compositions of the present invention in an amount of about 40% by weight, or about 50% by weight, or about 60% by weight, based on the total weight of the composition.

The composition of the present invention has a pH of at least about 9, preferably, greater than 11, more preferably, equal to or greater than 12, and can range from about 9 to about 14, such as from about 10 to about 14, preferably, from about 11 to about 14, more preferably from about 12 to about 14, and even more preferably from about 12.0 to about 13.6.

All numbers expressing pH values are to be understood as being modified in all instances by the term “about” which encompasses up to +/−3%. For example, a pH value of about 7.0 refers to 7+/−0.21.

The composition of the present invention has a viscosity ranging from about 50 uD to about 90 uD, such as from about 50 uD to about 80 uD, or such as from about 70 uD to about 80 uD or such as from about 50 uD to about 60 uD. Viscosity in uD (units of deflection) was measured by a Mettler RM 180 Rheomat, viscometer spindle #2, at 25° C.

In certain preferred embodiments, the composition of the present invention has a viscosity ranging from about 50 uD to about 90 uD and a pH ranging from about 9 to about 14.

In some preferred embodiments, the composition of the present invention has a viscosity ranging from about 70 uD to about 80 uD and a pH ranging from about 12.6 to about 13.6.

In other preferred embodiments, the composition of the present invention has a viscosity ranging from about 50 uD to about 60 uD and a pH ranging from about 12.14 to about 12.85.

In preferred embodiments, the composition of the present invention is a hair relaxing or straightening composition.

The composition of the present invention is stable such that the activity or efficacy of the alkaline material is preserved until the composition is ready to be used.

In addition, the composition of the present invention is stable over time; it can be stored for several months without modification and with very little change or fluctuation in the viscosity and/or rheology and pH of the composition.

In an embodiment, the present invention relates to a composition for relaxing or straightening hair, the composition comprising:
(a) from about 2% to about 6% by weight of active material of at least one alkaline material comprising at least one hydroxide-containing compound selected from alkali metal hydroxides, alkaline-earth metal hydroxides, transition metal hydroxides, and mixtures thereof;
(b) from about 1% to about 5% by weight of active material of at least one crosslinked copolymer of (meth)acrylic acid and/or (C1-C6)alkyl esters selected from a crosslinked (meth)acrylic acid/ethyl acrylate copolymer, a cross-linked anionic acrylate polymer, and mixtures thereof;
(c) a cosmetically acceptable solvent;
(d) from about 1% to about 15% by weight at least one surfactant selected from polysorbate-21, polysorbate-20, PEG-40 hydrogenated castor oil, PPG-5-Ceteth-20, caprylyl/capryl glucoside, decyl glucoside, and mixtures thereof; and
(e) from about 1% to about 5% by weight at least one thickening agent selected from acrylate- or acrylic-containing polymers other than (b), cellulose polymers, gums, silica material, starches, clay, and mixtures thereof;
   all weights above being based on the total weight of the composition;
   wherein the crosslinked copolymer (b) is fully or partially neutralized by about 0.5% to about 3% by weight at least one neutralizing agent;
   wherein the pH of the composition ranges from about 9 to about 14;
   wherein the viscosity of the composition is from about 50 uD to about 90 uD; and
   wherein the composition is clear in appearance.

In an embodiment, the present invention relates to a composition for relaxing or straightening hair, the composition comprising:
(a) from about 2% to about 6% by weight of active material of at least one alkaline material comprising at least one hydroxide-containing compound selected from alkali metal hydroxides, alkaline-earth metal hydroxides, transition metal hydroxides, and mixtures thereof;
(b) from about 1% to about 5% by weight of active material of at least one crosslinked copolymer of (meth)acrylic acid and/or (C1-C6)alkyl esters selected from a crosslinked (meth)acrylic acid/ethyl acrylate copolymer, a cross-linked anionic acrylate polymer, and mixtures thereof;
(c) a cosmetically acceptable solvent;
(d) from about 1% to about 15% by weight at least one surfactant selected from PEG-40 hydrogenated castor oil, PPG-5-Ceteth-20, caprylyl/capryl glucoside, decyl glucoside, and mixtures thereof;
(e) from about 40% to about 60% by weight at least one liquid fatty substance comprising mineral oil; and
(f) optionally, from about 1% to about 5% by weight at least one thickening agent selected from acrylate- or acrylic-containing polymers other than (b), cellulose polymers, gums, silica material, starches, clay, and mixtures thereof;
   all weights above being based on the total weight of the composition;
   wherein the crosslinked copolymer (b) is fully or partially neutralized by about 0.5% to about 3% by weight at least one neutralizing agent;
   wherein the pH of the composition ranges from about 9 to about 14;
   wherein the viscosity of the composition is from about 50 uD to about 90 uD; and
   wherein the composition is in the form of an emulsion.

The compositions of the present invention may be packaged in any suitable container such as a tube, a jar or a bottle. In certain embodiments, when the composition of the present invention is in the form of an aqueous gel or in the form of a cream, the composition can be packaged in a tube or bottle, for example, a squeeze tube or squeeze bottle or a drip tube or a drip bottle. The use of a tube or bottle allows for easier application of the product on the hair without having to scoop out the product from a jar or wide-mouthed containing and for easier transport of the product.

Additionally, an applicator device can be attached or connected to the opening of the tube or bottle wherein the applicator device can have teeth or a comb with teeth such that the ends of the teeth have openings from which the composition of the invention can flow through and be applied directly onto the hair.

When the composition of the present invention employs an alkaline material comprising calcium hydroxide, the composition may be packaged as first unit of a multi-compartment kit which contains a second unit comprising a composition containing an ingredient chosen from at least one carbonate compound. The compositions of the first and second units are mixed and resulting mixture or composition is applied onto hair in order to relax or straighten the hair.

The at least one carbonate compound of the present invention may be selected from lithium carbonate, sodium carbonate, potassium carbonate, guanidine carbonate, and mixtures thereof.

One particularly preferred carbonate compound is guanidine carbonate.

The at least one carbonate compound is employed in the compositions of the present invention in an amount ranging from about 1 to about 30% by weight, or preferably from about 5 to about 20% by weight, based on the total weight of the composition, including all ranges and subranges therebetween.

In some embodiments, the compositions of the present invention do not contain an oxidative dye precursor.

In other embodiments, the compositions of the present invention do not contain a direct dye.

In yet other embodiments, the compositions of the present invention do not contain an oxidizing agent chosen from peroxides, perborates, peroxygenated salts, ferricyanides, persulfates, bromates, percarbonates, lacasse enxymes and redox enzymes.

### Method of Relaxing or Straightening Hair

The invention also concerns a method of relaxing or straightening hair, the method comprising the steps of contacting hair with the composition of the invention, and 2) leaving the composition on the hair for a sufficient period of time to achieve a desired relaxation or straightening.

In accordance with the present invention, hair is contacted with the composition of the present invention in a sufficient amount as to desirably or effectively straighten or relax the hair.

In certain embodiments, upon contacting the hair with the composition and after a resting time (leave-on time) on the keratin fibers, for example, ranging from about 1 to about 60 minutes, such as from about 5 to about 45 minutes, or such as from about 5 to about 30 minutes, or such as from about 10 to about 20 minutes, or such as of about 20 minutes, the keratin fibers are rinsed, optionally washed with shampoo, rinsed again, optionally washed with a hair conditioning composition, and rinsed again, then dried, and/or styled, optionally, in the presence of heat, with a comb/brush/flat iron. Optionally, the hair can be smoothed by a smoothing action before, or while, or after contacting the hair with the composition of the present invention, using a device such as a brush, comb, or flat iron or by running the fingers through the hair. At the same time, heat (at a temperature of at least 37 degrees centigrade) can be applied to the hair while the smoothing action is performed on the hair. The heat source can be chosen from a blow dryer, a flat iron, or a hair dryer.

In addition, independently of the embodiment use, the composition present on the fibers or hair is left in place for a time, generally, from about 1 to about 60 minutes, such as from about 5 to about 45 minutes, or such as from about 5 to about 20 minutes, or such as from about 10 to about 20 minutes, or such as of about 20 minutes.

The temperature during the method of relaxing or straightening hair is between room temperature and about 80° C. and preferably, between room temperature and about 60° C.

It has been surprisingly and unexpectedly discovered that the compositions of the present invention have a non-drip consistency that is still easy to spread on keratin fibers, such as hair. This consistency is also characterized in terms of viscosity which was found to range from about 50 uD to about 90 uD.

It has surprisingly and unexpectedly discovered that the application of the composition onto the fibers results in satisfactory relaxing or straightening of hair.

The relaxing or straightening effects obtained using the compositions and method of the present disclosure may also be durable or wash resistant.

The degree of straightening or relaxing the hair may be evaluated by visually assessing the reduction in curliness and/or waviness and/or frizziness of the hair after contacting the hair with the composition of the invention. Another type of evaluation can also involve measuring the length of the hair as well as the width of the bulk of hair before and after contacting the hair with the composition.

It was surprisingly and unexpectedly discovered that the hair contacted with the compositions of the invention did not feel as rough and visually appeared to be more smooth, extended and straight compared to hair contacted with conventional or traditional straightening/relaxing compositions.

As used herein, the method and composition disclosed herein may be used on the hair that has not been artificially dyed, pigmented or permed.

As used herein, the method and composition disclosed herein may be also used on the hair that has been artificially dyed, pigmented or permed.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result.

### EXAMPLES

The following Examples are intended to be non-restrictive and explanatory only, with the scope of the invention being defined by the claims.

The ingredient amounts in the compositions/formulas described below are expressed in % by weight, based on the total weight of the composition/formula.

### Example 1

### Compositions

TABLE 1
Inventive Compositions

| | Formula A | Formula B |
|---|---|---|
| Ingredients/INCI names | % by weight | % by weight |
| Lithium Hydroxide | 4.00 | — |
| Sodium Hydroxide | — | 5.50 |
| Acrylates Copolymer* | 11.00 | 6.50 |
| AMP (2-amino-2-methyl-1-propanol) | 1.20 | 1.20 |
| Propylene Glycol** | 5.00 | 5.00 |
| Glycerin** | 2.00 | — |
| PPG-5-CETETH-20** | 4.00 | 3.00 |
| Carylyl/Capryl Glucoside** | 3.00 | 3.00 |
| Coco-Betaine** | 1.50 | — |
| Polyquaternium-39** | 1.20 | — |
| MINERAL OIL*** | — | 60.00 |
| Allantoin** | 1.20 | — |
| Polysobate-21** | 1.50 | — |
| Carbomer** | 4.00 | — |
| Sodium Polyacrylate** | 1.00 | 2.00 |
| Prunus Persica (Peach) Kernel Oil** | 0.17 | — |
| Ultramarine** | 0.03 | — |
| Deionized Water | QS 100 | QS 100 |

*(Carbopol ® Aqua SF-1, Lubrizol, 30% activity in water)
**optional ingredients
***mineral oil is an optional ingredient in formula A, a clear composition.

The compositions in Table 1 below were each prepared as follows: Formula A

1. Water was mixed with a chopper blade at about 250 rpm and the acrylates copolymer (Carbopol® Aqua SF-1) was added to the water. A neutralizing agent (e.g., AMP) was added to the solution and the solution was mixed for about 30 minutes and at a speed of from between about 150 rpm to about 300 rpm. The solution was additionally side swept for about 10 minutes or until the solution became clear.
2. Surfactants, conditioning agents and oils (PPG-5-ceteth-20, coco-betaine, polyquaternium-39, caprylyl/capryl glucoside, polysobate-21, allantoin) were each slowly added, one at a time, to the solution. The solution was side swept and mixed at at speed of from about 150 rpm to about 200 rpm. Mixing was continued for 10 minutes after each ingredient was added.
3. The alkaline material (e.g, lithium hydroxide) and organic solvent (e.g., propylene glycol, glycerin) were slowly added, one at a time, to the mixture. The solution was side swept and mixed at about 300 rpm. Mixing was continued for 10 minutes.
4. The thickening agent (e.g., carbomer, sodium polyacrylate) and colorant were slowly added, one at a time, to the solution. The solution was side swept and mixed at about 300 rpm. Mixing was continued for 10 minutes.

### Formula B

1. Water was mixed with a chopper blade at about 250 rpm and the acrylates copolymer, (Carbopol® Aqua SF-1) was added to the water. A neutralizing agent (e.g., AMP) was added to the solution and the solution was mixed for about 30 minutes and at a speed of from between about 150 rpm to about 300 rpm. The solution was additionally side swept for about 10 minutes or until the solution became clear.
2. One half of a liquid fatty substance (e.g., mineral oil) and surfactant (PPG-5-ceteth-20) were slowly added to the solution until it became pasty. The resulting pasty mixture was side swept and mixed at a speed of from about 800 rpm to about 1000 rpm. Mixing was continued for 10 minutes.
3. The alkaline material (e.g, sodium hydroxide) and organic solvent (e.g., propylene glycol) was slowly added to the mixture. The mixture was side swept and mixed at a speed of from about 800 rpm to about 1000 rpm. Mixing was continued for 10 minutes.
4. The remainder of the liquid fatty substance (mineral oil) was added to the mixture. The mixture was side swept and mixed at a speed of from about 800 rpm to about 1000 rpm. Mixing was continued for 10 minutes.
5. Another surfactant (e.g., caprylyl/capryl glucoside) was added to the mixture. The mixture was side swept and mixed at a speed of from about 800 rpm to about 1000 rpm. Mixing was continued for 10 minutes.
6. The thickening agent (e.g. sodium polyacrylate) was slowly added to the solution. The solution was side swept and mixed at about 300 rpm. Mixing was continued for 10 minutes.

Formula A was a gel and was observed to be clear in appearance and formula B was a cream emulsion.

Formulas C and D were also prepared; they were similar to Formulas A and B, except that Formula C used a gelling agent as the optional thickening agent and Formula D employed 8% by weight of acrylates copolymer, Carbopol® Aqua SF-1 dispersion (30% active), 40% by weight of mineral oil, and a gelling agent as the optional thickening agent. Formula C was in gel form and clear in appearance and formula D was a cream emulsion. The formulas A to D had smooth textures and were observed not to be sticky and not greasy, even when oil was present in high amounts in formulas B and D. The compositions also had non-drip consistencies, i.e, not runny or watery.

A fifth formula, E, contained 6.5% by weight of acrylates copolymer, Carbopol® Aqua SF-1 dispersion, 30% active pre-neutralized by 0.5% by weight of sodium hydroxide, 60% by weight of mineral oil, 5.0% by weight of sodium hydroxide, 5% propylene glycol and water (Q.S.), all weights based on the total weight of the composition.

The consistency and texture of the inventive formulas also allow for the formulas to be packaged in tubes (e.g., squeeze tubes) or bottles (eg, applicator bottles).

TABLE 2
Comparative compositions

| Comparative | |
|---|---|
| compositions | Key Ingredients |
| Formula 1 | Calcium hydroxide, petrolatum and mineral oil (32% by |
| | weight), cetyl alcohol, water, cetearyl alcohol (and) |
| | polysorbate 60, peg-75 lanolin, |
| Formula 2 | Sodium hydroxide, petrolatum and mineral oil (32% by |
| | weight), hexylene glycol, water, cetearyl alcohol (and) |
| | polysorbate 60, peg-75 lanolin |
| Formula 3 | Sodium hydroxide, petrolatum and mineral oil (32% by |
| | weight), polyquaternium-6, water, propylene glycol, |
| | cetearyl alcohol (and) behentrimonium methosulfate, |
| | cetearyl alcohol (and) polysorbate 60, peg-75 lanolin |

The comparative formulas above are conventional formulas in thick, heavy paste form (not in cream form) and contained in jars or wide-mouthed containers (not in tubes). These formulas were observed to be sticky.

### Example 2

### Viscosity and pH Studies Over Time

The viscosities and the pH of the compositions of the invention were measured and also compared to the viscosities and pH of the comparative compositions. Viscosity was measured using the Mettler RM 180 Rheomat, viscometer spindle #2, at 25° C. (uD=Units of Deflation).

The viscosities and pH values for the test compositions studied were measured at room temperature over various time points in order to determine whether the viscosity and pH of the test compositions changed over a period of 60 minutes with constant mixing using a Rayneri mixer.

TABLE 3
Viscosity study; Inventive composition versus comparative compositions

| | Viscosity* | | | | |
|---|---|---|---|---|---|
| | | | | Inventive | Inventive |
| | Comparative | Comparative | Comparative | com- | com- |
| Time | composition | composition | composition | position | position |
| (min) | Formula 1 | Formula 2 | Formula 3 | Formula C | Formula D |
| 0 | 45.40 | 39.60 | 43.90 | 76.35 | 71.15 |
| 5 | 46.30 | 38.60 | 42.15 | 76.35 | 71.15 |
| 10 | 44.80 | 39.17 | 41.36 | 76.45 | 75.14 |
| 15 | 46.12 | 39.15 | 41.35 | 76.45 | 71.15 |
| 20 | 46.14 | 38.12 | 42.15 | 76.19 | 70.69 |
| 25 | 42.36 | 38.96 | 44.15 | 76.89 | 70.25 |
| 30 | 41.15 | 38.00 | 39.01 | 75.15 | 70.01 |
| 35 | 39.35 | 33.65 | 36.52 | 75.14 | 68.00 |
| 40 | 38.60 | 32.15 | 39.46 | 75.05 | 68.00 |
| 45 | 37.50 | 31.15 | 39.78 | 75.01 | 69.15 |
| 50 | 33.12 | 30.00 | 30.12 | 74.32 | 68.15 |
| 55 | 31.25 | 29.45 | 24.65 | 73.15 | 68.15 |
| 60 | 25.90 | 25.25 | 23.12 | 72.19 | 66.21 |

*In the viscosity measurements, for the compositions of the invention, viscometer spindle 2 was used and for the comparative compositions, viscometer spindle 4 was used because of their thick, heavy paste consistencies.

The results in the table above show that the changes in viscosities over time of the compositions of the invention were smaller compared to the changes in viscosities over time of the comparative compositions.

Formula A had a viscosity of 59.6 uD and a pH of 12.14, and formula B had a viscosity of 64.1 uD and a pH of 12.85. The changes in viscosities over time for these two formulas were comparable to those observed for Formulas C and D.

The measured viscosities can also be associated with the smooth textures of the inventive compositions (A to D) and a creamy texture for formulas B and D, with non-sticky properties and with non-drip consistencies, i.e, they were not runny or watery.

TABLE 4
pH study

| | pH | | | | |
|---|---|---|---|---|---|
| | | | | Inventive | Inventive |
| | Comparative | Comparative | Comparative | com- | com- |
| Time | composition | composition | composition | position | position |
| (min) | Formula 1 | Formula 2 | Formula 3 | Formula C | Formula D |
| 0 | 13.12 | 13.23 | 12.99 | 13.10 | 12.85 |
| 5 | 13.14 | 13.25 | 12.90 | 13.10 | 12.85 |
| 10 | 13.15 | 13.20 | 12.91 | 13.10 | 12.80 |
| 15 | 13.26 | 13.15 | 12.90 | 13.05 | 12.79 |
| 20 | 12.99 | 13.15 | 12.89 | 13.01 | 12.75 |
| 25 | 12.95 | 13.25 | 12.89 | 13.01 | 12.76 |
| 30 | 13.01 | 13.19 | 12.91 | 12.98 | 12.70 |
| 35 | 13.20 | 13.19 | 12.89 | 12.96 | 12.69 |
| 40 | 13.25 | 13.20 | 12.90 | 12.95 | 12.65 |
| 45 | 13.25 | 13.25 | 12.95 | 12.89 | 12.50 |
| 50 | 13.23 | 13.26 | 12.96 | 12.89 | 12.59 |
| 55 | 13.02 | 13.25 | 12.98 | 12.89 | 12.58 |
| 60 | 13.00 | 13.25 | 12.99 | 12.95 | 12.56 |

It was found that the pH values of the compositions of the invention were comparable to the pH of the comparative compositions and also remained substantially the same over time. This shows that the inventive compositions have a pH that is desirable for achieving effective straightening or relaxation of hair.

### Example 3

### Stability of Inventive Compositions

The inventive compositions were shown to be stable up to 8 weeks in a controlled chamber at various temperatures at 5° C., 25° C., 37° C., and 45° C. as well as 10 days in a Freeze/Thaw cycle where the pH and viscosity had small fluctuations and there was no phase separation.

The viscosity/consistency of the inventive compositions were observed to remain the same even after 8 weeks.

### Example 4

### Relaxation or Straightening Studies on Hair

Designated swatches of curly, virgin (not chemically treated) hair were contacted with the test composition, Formula A (invention). Application of the test composition was conducted as follows: 10 grams of a composition was applied onto a swatch, massaged into the hair and left on the hair for about 15 minutes. The hair was then washed with a neutralizing shampoo, rinsed with water, and then combed or brushed, and blow-dried.

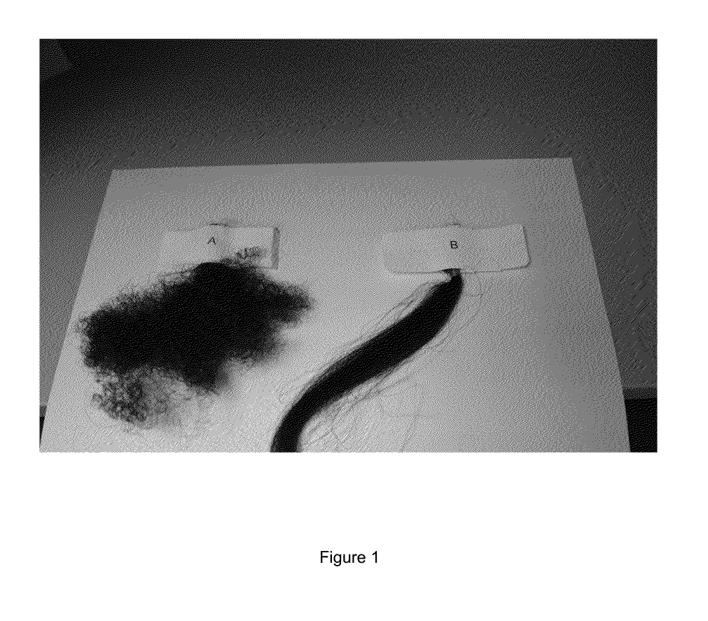

Photographic images of the hair swatches (FIG. 1) were taken and visually assessed for the straightening/relaxing effects imparted by the test compositions. In FIG. 1, the first swatch (control) on the left (A) was shampooed, rinsed, combed or brushed, and blow-dried but not contacted with any test composition. The second swatch (B) was contacted with formula A.

A visual evaluation of the swatches on the photographic image, shows that the composition of the invention significantly and effectively straightened/relaxed the hair.

Similar tests using formulas B, C and D (invention), on curly, virgin (not chemically treated) hair yielded the same degree of straightening as formula A.

It is to be understood that the foregoing describes preferred embodiments of the invention and that modifications may be made therein without departing from the spirit or scope of the invention as set forth in the claims.

What is claimed is:

1. A composition comprising:
(a) at least one alkaline material comprising at least one hydroxide-containing compound selected from alkali metal hydroxides, alkaline-earth metal hydroxides, transition metal hydroxides, and mixtures thereof;
(b) at least one crosslinked copolymer of (meth)acrylic acid and/or (C1-C6)alkyl esters selected from a crosslinked (meth)acrylic acid/ethyl acrylate copolymer, a cross-linked anionic acrylate polymer, and mixtures thereof; and
(c) a cosmetically acceptable solvent;
wherein the pH of the composition ranges from about 9 to about 14; and
wherein the viscosity of the composition is from about 50 uD to about 90 uD.

2. The composition of claim 1, wherein the alkaline material is present in an amount of from about 1% to about 30% by weight of active material, based on the total weight of the composition.

3. The composition of claim 2, wherein the hydroxide-containing compound is selected from sodium hydroxide, potassium hydroxide, lithium hydroxide, calcium hydroxide, magnesium hydroxide, barium hydroxide, strontium hydroxide, manganese hydroxide, zinc hydroxide, and mixtures thereof.

4. The composition of claim 3, wherein the crosslinked copolymer (b) is present in an amount of from about 0.5% to about 15% by weight of active material, based on the total weight of the composition.

5. The composition of claim 4, wherein the crosslinked copolymer (b) is crosslinked (meth)acrylic acid/ethyl acrylate copolymer.

6. The composition of claim 5, wherein the crosslinked (meth)acrylic acid/ethyl acrylate copolymer is an acrylates copolymer contained in an aqueous dispersion; wherein the aqueous dispersion comprises about 30% by weight of the acrylates copolymer, based on the weight of the dispersion.

7. The composition according to claim 4, wherein the crosslinked copolymer (b) is a cross-linked anionic acrylate polymer.

8. The composition according to claim 7, wherein the cross-linked anionic acrylate polymer is acrylates crosspolymer-4.

9. The composition of claim 4, wherein the crosslinked copolymer (b) is fully or partially neutralized by at least one neutralizing agent selected from alkali metal carbonates, alkali metal phosphates, organic amines, hydroxide base compounds, and mixtures thereof.

10. The composition of claim 9, wherein the at least one neutralizing agent is employed in an amount of from about 0.01% to less than about 5% by weight, based on the total weight of the composition.

11. The composition of claim 4, wherein the cosmetically acceptable solvent is selected from water, an organic solvent, and mixtures thereof.

12. The composition of claim 11, further comprising at least one liquid fatty substance.

13. The composition of claim 12, wherein the liquid fatty substance is selected from C₆-C₁₆ alkanes, non-silicone oils of plant, mineral or synthetic origin, liquid fatty alcohols, liquid fatty acids and liquid esters of a fatty acid and/or of a fatty alcohol, or mixtures thereof.

14. The composition of claim 13, wherein the liquid fatty substance is present in an amount of from about 10% to about 60% by weight, based on the total weight of the composition.

15. The composition of claim 11, further comprising at least one thickening agent selected from polymeric compounds other than (b), non-polymeric compounds, and mixtures thereof.

16. The composition of claim 15, wherein the thickening agent is selected from acrylate- or acrylic-containing polymers, cellulose polymers, gums, silica material, starches, clay, and mixtures thereof.

17. The composition of claim 16, wherein the thickening agent is present in an amount of from about 0.1% to about 10% by weight, based on the total weight of the composition.

18. The composition of claim 11, wherein the hydroxide-containing compound (a) is calcium hydroxide and the composition further comprises at least one carbonate compound selected from lithium carbonate, sodium carbonate, potassium carbonate, guanidine carbonate, and mixtures thereof.

19. The composition of claim 11, wherein the composition further comprises at least one surfactant selected from anionic surfactants, nonionic surfactants, amphoteric surfactants, and mixtures thereof.

20. The composition of claim 11, further comprising at least one auxiliary ingredient selected from emulsifying agents, conditioning agents, moisturizing agents, shine agents, fillers, colorants, pigments, chelating agents, sequestering agents, fragrances, preservatives, stabilizers, and mixtures thereof.

21. A method of relaxing or straightening hair, comprising contacting hair with the composition of claim 1.

22. A composition for relaxing or straightening hair comprising:
(a) from about 1% to about 20% by weight of active material of at least one alkaline material comprising at least one hydroxide-containing compound selected from alkali metal hydroxides, alkaline-earth metal hydroxides, transition metal hydroxides, and mixtures thereof;
(b) from about 1% to about 12% by weight of active material of at least one crosslinked copolymer of (meth)acrylic acid and/or (C1-C6)alkyl esters selected from a crosslinked (meth)acrylic acid/ethyl acrylate copolymer, a cross-linked anionic acrylate polymer, and mixtures thereof;
(c) a cosmetically acceptable solvent;
(d) from about 1% to about 20% by weight at least one surfactant selected from polysorbate-21, polysorbate-20, PEG-40 hydrogenated castor oil, PPG-5-Ceteth-20, caprylyl/capryl glucoside, decyl glucoside, and mixtures thereof; and
(e) from about 0.5% to about 15% by weight at least one thickening agent selected from acrylate- or acrylic-containing polymers other than (b), cellulose polymers, gums, silica material, starches, clay, and mixtures thereof;
all weights above being based on the total weight of the composition;
wherein the crosslinked copolymer (b) is fully or partially neutralized by at least one neutralizing agent;
wherein the pH of the composition ranges from about 9 to about 14;
wherein the viscosity of the composition is from about 50 uD to about 90 uD; and
wherein the composition is clear in appearance.

23. A composition for relaxing or straightening hair comprising:
(a) from about 1% to about 20% by weight of active material of at least one alkaline material comprising at least one hydroxide-containing compound selected from alkali metal hydroxides, alkaline-earth metal hydroxides, transition metal hydroxides, and mixtures thereof;
(b) from about 1% to about 12% by weight of active material of at least one crosslinked copolymer of (meth)acrylic acid and/or (C1-C6)alkyl esters selected from a crosslinked (meth)acrylic acid/ethyl acrylate copolymer, a cross-linked anionic acrylate polymer, and mixtures thereof;
(c) a cosmetically acceptable solvent;
(d) from about 1% to about 20% by weight at least one surfactant selected from PEG-40 hydrogenated castor oil, PPG-5-Ceteth-20, caprylyl/capryl glucoside, decyl glucoside, and mixtures thereof;
(e) from about 10% to about 60% by weight at least one liquid fatty substance; and
(f) optionally, from about 0.5% to about 15% by weight at least one thickening agent selected from acrylate- or acrylic-containing polymers other than (b), cellulose polymers, gums, silica material, starches, clay, and mixtures thereof;
all weights above being based on the total weight of the composition;
wherein the crosslinked copolymer (b) is fully or partially neutralized by at least one neutralizing agent;
wherein the pH of the composition ranges from about 9 to about 14;
wherein the viscosity of the composition is from about 50 uD to about 90 uD; and
wherein the composition is in the form of an emulsion.

24. A method of relaxing or straightening hair, the method comprising:
1) contacting hair with a composition containing:
(a) from about 1% to about 30% by weight of active material of at least one alkaline material comprising at least one hydroxide-containing compound selected from alkali metal hydroxides, alkaline-earth metal hydroxides, transition metal hydroxides, and mixtures thereof;
(b) from about 0.5% to about 15% by weight of active material of at least one crosslinked copolymer of (meth)acrylic acid and/or (C1-C6)alkyl esters selected from a crosslinked (meth)acrylic acid/ethyl acrylate copolymer, a cross-linked anionic acrylate polymer, and mixtures thereof;
(c) a cosmetically acceptable solvent;
(d) optionally, from about 0.01% to about 40% by weight at least one surfactant;
(e) optionally, from about 10% to about 60% by weight at least one liquid fatty substance;
(f) optionally, from about 0.1% to about 20% by weight at least one thickening agent selected from acrylate- or acrylic-containing polymers other than (b), cellulose polymers, gums, silica material, starches, clay, and mixtures thereof; and
all weights above being based on the total weight of the composition;
wherein the crosslinked copolymer (b) is fully or partially neutralized by at least one neutralizing agent;
wherein the pH of the composition ranges from about 9 to about 14;
wherein the viscosity of the composition is from about 50 uD to about 90 uD; and
2) leaving the composition on the hair for a sufficient period of time to achieve a desired relaxation or straightening.
